(19) United States
(12) Patent Application Publication
(10) Pub. No.: US 2024/0261382 A1
(43) Pub. Date: Aug. 8, 2024

(54) MODIFIED BACTERIUM, PREPARATION METHOD THEREFOR AND APPLICATION THEREOF

(71) Applicant: INNOBM PHARMACEUTICALS CO., LTD., Suzhou, Jiangsu (CN)

(72) Inventors: Zhuang LIU, Suzhou, Jiangsu (CN); Rui PENG, Suzhou, Jiangsu (CN); Chenya WANG, Suzhou, Jiangsu (CN); Qi ZHUANG, Suzhou, Jiangsu (CN); Qi ZHAO, Suzhou, Jiangsu (CN)

(21) Appl. No.: 18/567,058

(22) PCT Filed: Jun. 6, 2022
(86) PCT No.: PCT/CN2022/097190

§ 371 Date: Dec. 5, 2023

(30) Foreign Application Priority Data
Jun. 7, 2021 (CN) 202110631604.6
Oct. 26, 2021 (CN) 202111249964.6
Nov. 1, 2021 (CN) 202111284429.4

(51) Int. Cl. A61K 39/112; A61K 39/00; A61K 39/085; A61K 39/09; A61K 39/108; A61P 35/00; C12N 1/20

(52) U.S. Cl. CPC A61K 39/0275; A61K 39/0258; A61K 39/085; A61K 39/09; A61P 35/00; C12N 1/20; A61K 2039/521; A61K 2039/55505; A61K 2039/585

(57) ABSTRACT

A modified bacterium, a preparation method thereof and an application thereof. The modified bacterium comprises a bacterium body and a poorly soluble or insoluble biologically acceptable metal compound modified on the surface of the bacterium body. After the modified bacterium is injected in a tumor, an anti-tumor immune response can be activated, an excellent anti-tumor treatment is activated, and tumor metastasis and recurrence can be effectively inhibited.

### TECHNICAL FIELD

The present application relates to the field of biomedicine, in particular to a modified bacterium, a preparation method thereof and an application thereof.

### BACKGROUND

Bacteria-based cancer therapy has a long history, but its development is slow. It was not until the 1990s, when the BCG vaccine was used to treat human bladder cancer, that bacterial therapy was introduced to the public again. In recent years, attenuated bacteria such as attenuated Salmonella and attenuated Listeria have been gradually developed for the treatment of different tumors. Bacterial therapy for tumors is also an extension of immunotherapy, which uses the stimulation of bacteria, a heterologous foreign substance, to trigger an immune response and further inhibit tumor growth. However, most of the strains currently used are attenuated living bacteria that still carry high risks in clinical use and have a narrow safety window. Inactivated bacteria cannot achieve the desired immune stimulating effect. Generally speaking, it is difficult for current tumor bacterial therapies to achieve effective treatment at safe doses.

As a transition metal element and one of the trace elements in the human body, manganese has been gradually used in some patents to explore its application in immune stimulation enhancement. For example, patents CN201610347815.6 and CN201910319344.1 disclose the function of activation function of manganese in the STING pathway, and manganese precipitate or colloidal manganese has an immune-enhancing effect. Among them, the '156 patent discloses that divalent manganese can activate the STING pathway, but the immune-enhancing effect of divalent manganese is undesirable and it is difficult to obtain a high concentration of divalent manganese to enhance the effect; in the '441 patent, divalent nascent precipitated manganese and colloidal manganese are used to stimulate the immune system, a stronger immune stimulating effect is obtained than divalent manganese ions, and subsequently, the nascent precipitated manganese, colloidal manganese and an antigen are simply mixed to obtain a vaccine that's more effective than the antigen. Additionally, patent CN201710795715.4 discloses a manganese dioxide nanoparticle, where oligonucleotides (CpG) and/or antigens are loaded on the surface of the manganese dioxide nanoparticles to synergistically enhance the immune stimulating effect, and as manganese dioxide degrades in a weakly acidic environment, manganese ions can eventually be excreted from the body. Utilizing the adjuvant-like function of manganese ions, it can activate the STING pathway, induce the production of type I interferon to regulate the host immune system, activate T cell-specific immune responses, and induce macrophages to polarize toward an anti-tumor phenotype to induce pathogen-specific immune responses, thereby further inducing the generation of immune memory. However, in the treatment strategies of the above-mentioned patents, manganese ions function as immune adjuvants, and similar to aluminum adjuvants, an antigen must be added to induce an immune response against this antigen, and it plays the role of a vaccine. For example, in the '441 patent, manganese ions (or other manganese-containing substances) are mixed with inactivated bacteria and then injected, which mainly produces an immune response against the bacteria to achieve the purpose of preventing bacterial infection; if manganese ions (or other manganese-containing substances) are mixed with a mutated antigen specifically expressed by tumor cells and then injected, what is generated is an immune response against the antigen, which will produce immune attack against tumor cells that express this antigen, but will be ineffective against tumor cells that do not express this antigen.

### SUMMARY

The present application provides a modified bacterium, a preparation method thereof and an application thereof. The modified bacterium has one or more of the following advantages: (1) the present application provides a modified bacterium that can be an inactivated bacterium, the metal compounds on its surface can be metabolized by being decomposed into an ionic state, which has better safety; (2) the insoluble metal compounds modified on the surface of the bacterium provided by the present application can neutralize the weak acidic microenvironment of tumors, can increase the activity of immune cells at tumor sites, reduce tumor drug resistance, and improve efficacy; (3) the modified bacterium provided by the present application is a new type of immune agonist, which can activate immune cells through stimulation of multiple pathways to induce strong anti-tumor immune responses and produce immune memory effects to reduce the probability of cancer metastasis and recurrence.

In one aspect, the present application provides a modified bacterium comprising a bacterium body and a poorly soluble or insoluble biologically acceptable metal compound modified on the surface of the bacterium body.

In another aspect, the present application provides a modified bacterium comprising a bacterium body and a poorly soluble or insoluble biologically acceptable metal compound modified on the surface of the bacterium body, wherein the metal compound is modified on the surface of the bacterium body to form an adhesion layer through deposition.

In another aspect, the present application provides a modified bacterium comprising a bacterium body and a poorly soluble or insoluble biologically acceptable metal compound modified on the surface of the bacterium body, wherein the content of the metal compound on every 1*10⁸ bacteria is about 0.1 μg to 600 μg.

In some embodiments, the bacterium body is a Gram-positive bacterium or a Gram-negative bacterium.

In some embodiments, the bacterium body is selected from one or more of coccus,

bacillus, and spirillum.

In some embodiments, the coccus is selected from one or more of Staphylococcus aureus, Micrococcus urea, Diplococcus pneumoniae, Streptococcus pneumoniae, Diplococcus meningitidis, Streptococcus pyogenes, Streptococcus pneumoniae, Streptococcus lactis, Staphylococcus aureus, Staphylococcus albus, and Staphylococcus citreus.

In some embodiments, the bacillus is selected from one or more of Escherichia coli, Salmonella, Yersinia pestis, Bacillus dysenteriae suis, Pasteurella multocida, Corynebacterium diphtheriae, Mycobacterium tuberculosis Bacillus, Lactobacillus bifidum, Bacillus aceticus, and Corynebacterium.

In some embodiments, the spirillum is selected from one or more of Helicobacter pylori, Vibrio comma and Vibrio cholerae.

In some embodiments, the bacterium body is selected from one or more of Salmonella, Staphylococcus aureus, Escherichia coli, Lactobacillus, attenuated strains of Salmonella, attenuated strains of Staphylococcus aureus, attenuated strains of Escherichia coliand attenuated strains of Lactobacillus.

In some embodiments, the bacterium body is a wild-type strain, a genetically engineered strain, and/or an attenuated strain.

In some embodiments, the bacterium body is a living bacterium or an inactivated bacterium.

In some embodiments, the living bacteria or inactivated bacteria includes attenuated bacteria.

In some embodiments, the metal compound is modified on the surface of the bacterium body to form an adhesion layer through physical binding or chemical binding.

In some embodiments, the physical binding includes electrostatic adsorption and/or embedded.

In some embodiments, the chemical binding includes coupling, forming chemical bonds and/or complexation.

In some embodiments, the metal compound is modified on the surface of the bacterium body to form an adhesion layer through deposition.

In some embodiments, the metal compound is modified on the surface of the bacterium body to form an adhesion layer through biomineralization.

In some embodiments, the biomineralization comprise: metal ions contact with biomacromolecules on the cell membrane or the cell wall of bacteria to provide mineralization sites, pH is adjusted or other salts are introduced, the metal ions generate metal compounds at the mineralization sites, the metal compounds continue to grow and accumulate and bind to the surface of bacteria.

In some embodiments, binding sites for biomacromolecules and metal compounds are formed through the biomineralization, and the volume of the metal compound is further increased by deposition or biomineralization.

In some embodiments, the coverage of the metal compound on the surface of the bacterium is 0.1%-99.9%.

In some embodiments, the coverage of the metal compound on the surface of the bacterium is adjusted by adjusting feed ratio, reaction temperature and/or reaction time.

In some embodiments, the metal compound has a metal element selected from one or more of zinc, calcium, copper, iron, manganese and magnesium; and a non-metal element selected from one or more of carbonate radical, hydroxide radical, sulfide radical and phosphate radical.

In some embodiments, the metal compound is selected from one or more of zinc carbonate, calcium carbonate, copper carbonate, magnesium carbonate; zinc hydroxide, ferric hydroxide, copper hydroxide, manganese hydroxide, magnesium hydroxide, zinc sulfide, copper sulfide, manganese sulfide; zinc phosphate, calcium phosphate, copper phosphate, iron phosphate, magnesium phosphate and manganese phosphate.

In some embodiments, the metal compound is a manganese-containing compound.

In some embodiments, the manganese-containing compound is one or more of manganese hydroxide, manganese dioxide and manganese sulfide. In some embodiments, the manganese-containing compound is manganese hydroxide and/or manganese dioxide.

In another aspect, the present application provides a manganese-biomineralized bacterium comprising a bacterium body and a poorly soluble or slightly soluble manganese-containing compound adhering to the surface of the bacterium body.

In some embodiments, the poorly soluble or slightly soluble manganese-containing compound is one or more of manganese hydroxide, manganese dioxide and manganese sulfide.

In some embodiments, the bacterium body is a Gram-positive bacterium or a Gram-negative bacterium.

In some embodiments, the bacterium body is selected from coccus, bacillus, and spirillum.

In some embodiments, the coccus is selected from one or more of Staphylococcus aureus, Micrococcus urea, Diplococcus pneumoniae, Streptococcus pneumoniae, Diplococcus meningitidis, Streptococcus pyogenes, Streptococcus pneumoniae, Streptococcus lactis, Staphylococcus aureus, Staphylococcus albus, and Staphylococcus citreus.

In some embodiments, the bacillus is selected from one or more of Escherichia coli, Salmonella, Yersinia pestis, Bacillus dysenteriae suis, Pasteurella multocida, Corynebacterium diphtheriae, Mycobacterium tuberculosis Bacillus, Lactobacillus bifidum, Bacillus aceticus, and Corynebacterium.

In some embodiments, the spirillum is selected from one or more of Helicobacter pylori, Vibrio comma and Vibrio cholerae.

In some embodiments, the bacterium body is a living bacterium or an inactivated bacterium.

In some embodiments, the bacterium is a wild-type strain, a genetically engineered strain, and/or an attenuated strain.

In another aspect, the present application provides a composition comprising the modified bacterium or the manganese-biomineralized bacterium, and optionally a pharmaceutically acceptable carrier, wherein the modified bacterium comprises a bacterium body and a poorly soluble or insoluble biologically acceptable metal compound modified on the surface of the bacterium body, and the pH value of the composition is 0-14.

In another aspect, the present application provides a modified bacterial freeze-dried powder comprising the modified bacterium and an additive.

In some embodiments, the additive includes freeze-drying protective agents and/or excipients.

In some embodiments, the freeze-drying protective agent is selected from one or more of sugars/polyols, polymers, anhydrous solvents, surfactants, amino acids, salts and amines.

In some embodiments, the excipient is selected from one or more of binders, fillers, disintegrants, lubricants, wine, vinegar, decoctions, etc., ointment bases, cream bases, preservatives, antioxidants, flavoring agents, fragrances, co-solvents, emulsifiers, solubilizers, osmotic pressure regulators and colorants.

In some embodiments, the mass fraction of the additive in the modified bacterial freeze-dried powder is 0.1-99%. The mass fraction here is defined as the mass fraction of the freeze-drying protective agent in the freeze-dried product.

In some embodiments, the freeze-drying protective agent is selected from one or more of sucrose, mannose, a-D-mannopyranose, trehalose, inositol, raffinose, inulin, dextran, maltodextrin, maltopolysaccharide, sucrose octasulfate, heparin and 2-hydroxypropyl-β-cyclodextrin.

In some embodiments, the mass fraction of the freeze-drying protective agent in the bacterial freeze-dried powder is 0.1-99%. The mass fraction here is defined as the mass fraction of the freeze-drying protective agent in the freeze-dried product.

In another aspect, the present application provides a manganese-biomineralized bacterial freeze-dried powder comprising the manganese-biomineralized bacterium and an additive.

In some embodiments, the additive includes freeze-drying protective agents and/or excipients.

In some embodiments, the freeze-drying protective agent is selected from one or more of sugars/polyols, polymers, anhydrous solvents, surfactants, amino acids, salts and amines.

In some embodiments, the excipient is selected from one or more of binders, fillers, disintegrants, lubricants, wine, vinegar, decoctions, etc., ointment bases, cream bases, preservatives, antioxidants, flavoring agents, fragrances, co-solvents, emulsifiers, solubilizers, osmotic pressure regulators and colorants.

In some embodiments, the mass fraction of the additive in the bacterial freeze-dried powder is 0.1-99%. The mass fraction here is defined as the mass fraction of the freeze-drying protective agent in the freeze-dried product.

In some embodiments, the freeze-drying protective agent is selected from one or more of sucrose, mannose, a-D-mannopyranose, trehalose, inositol, raffinose, inulin, dextran, maltodextrin, maltopolysaccharide, sucrose octasulfate, heparin and 2-hydroxypropyl-β-cyclodextrin.

In some embodiments, the mass fraction of the freeze-drying protective agent in the bacterial freeze-dried powder is 0.1-99%. The mass fraction here is defined as the mass fraction of the freeze-drying protective agent in the freeze-dried product.

In another aspect, the present application provides a preparation method of the modified bacterium or the manganese-biomineralized bacterium, which includes the following steps: S1: preparation of a bacterial suspension; S2: adding a soluble metal salt solution to the bacterial suspension.

In some embodiments, the preparation method further comprises step S3: adding an easily soluble aqueous hydroxide solution, aqueous carbonate solution or aqueous phosphate solution to reach a pH of 8-12, or adding an easily soluble aqueous sulfide solution to react to prepare the modified bacterium.

In some embodiments, in the preparation method, the easily soluble aqueous sulfide solution includes a sodium sulfide solution, a potassium sulfide solution and/or an ammonium sulfide solution.

In some embodiments, in the preparation method, the easily soluble aqueous hydroxide solution, aqueous carbonate solution or aqueous phosphate solution is selected from: an aqueous sodium hydroxide solution, an aqueous sodium carbonate solution, and an aqueous sodium phosphate solution.

In some embodiments, in the step S2 of the preparation method, a soluble metal salt solution is added to the bacterial suspension at an amount of 0.2-13.5 mmol of metal ions per 1 billion bacteria.

In some embodiments, in the preparation method, the soluble metal salt solution is a permanganate solution.

In some embodiments, the step S2 of the preparation method comprises adding the permanganate solution to the bacterial suspension, reacting with stirring, and centrifuging to prepare manganese-biomineralized bacteria.

In some embodiments, in the preparation method, 0.2-3 umol of permanganate is added per 1 billion bacteria.

In another aspect, the present application provides use of the modified bacterium, the manganese-biomineralized bacterium, the modified bacterial freeze-dried powder, the manganese-biomineralized bacterial freeze-dried powder, or the composition in the preparation of tumor immunotherapeutics.

In another aspect, the present application provides the modified bacterium, the manganese-biomineralized bacterium, the modified bacterial freeze-dried powder, the manganese-biomineralized bacterial freeze-dried powder, or the composition for use in the prevention and/or treatment of tumors.

In another aspect, the present application provides a method for preventing and/or treating tumors, which comprises administering to a subject in need thereof an effective amount of the modified bacterium, the manganese-biomineralized bacterium, the modified bacterial freeze-dried powder, the manganese-biomineralized bacterial freeze-dried powder, or the composition.

Those skilled in the art will readily appreciate other aspects and advantages of the present application from the detailed description below. Only exemplary embodiments of the present application are shown and described in the following detailed description. As those skilled in the art will realize, the contents of the present application enable those skilled in the art to make changes to the specific embodiments disclosed without departing from the spirit and scope of the invention covered by the present application. Accordingly, the descriptions of the drawings and specification of the present application are illustrative only and not restrictive.

### BRIEF DESCRIPTION OF THE DRAWINGS

The specific features of the invention to which the present application relates are set forth in the appended claims. The features and advantages of the invention to which the present application relates can be better understood by reference to the exemplary embodiments described in detail below and the drawings. A brief description of the drawings is as follows:

FIG. 1 shows SEM images of modified inactivated bacteria adhered with different metal compounds;

FIG. 2 shows SEM images of different bacteria before and after modification with manganese dioxide;

FIG. 3 is a statistical diagram of the relative activity of CT26 cells after co-incubation with different samples for 12 h;

FIG. 4 is a statistical diagram of the proportion of mature cells detected by flow cytometry after co-incubation of bone marrow-derived dendritic cells with different samples;

FIG. 5 shows the tumor growth curves of tumor-bearing mice after treating with bacteria modified with different metal compounds.

FIG. 6 is a statistical diagram of the bioluminescence signal intensity of reporter gene expression after the STING pathway is activated in different groups after incubation of reporter cells with different samples for 24 h;

FIG. 7 shows the survival rate curves of mice inoculated with the same tumor again on the 60th day after the mouse colon cancer subcutaneous tumor model was cured by inactivated Salmonella modified with manganese dioxide.

FIG. 8 shows the XPS spectra of different bacteria after manganization;

FIG. 9 is a statistical diagram of the intracellular Cy5.5 signal intensity detected by flow cytometry at different time points after co-incubation of DC2.4 cells with Cy5.5-labeled bacteria (F.S) or Cy5.5-labeled manganese dioxide-biomineralized bacteria (MnO_{2@}F.S);

FIG. 10 is a statistical diagram of the content of manganese ions phagocytosed in the cells after co-incubation of DC2.4 cells with manganese dioxide (MnO₂) or manganese dioxide-biomineralized bacteria (MnO₂@F.S);

FIG. 11 is a statistical diagram of the proportion of mature dendritic cells detected after co-incubation of bone marrow-derived dendritic cells (BMDCs) with different samples for 12 h;

FIG. 12 is a statistical diagram of the concentration of cytokines in the supernatant of the culture medium detected after co-incubation of bone marrow-derived dendritic cells (BMDCs) with different samples for 12 h;

FIG. 13 is a statistical diagram of the concentration of cytokines in the culture medium after co-incubation of peritoneal macrophages with different samples;

FIG. 14 shows a western blot diagram of interferon regulatory factor (IRF3), phosphorylated interferon regulatory factor (p-IRF3) and a statistical diagram of the corresponding IRF3 phosphorylation rate after incubation of reporter cells with different samples for 24 h;

FIG. 15 is a statistical diagram of the downstream fluorescence signal intensity after Toll-like receptor 4 activation after incubation of Toll-like receptor 4 reporter cells with different samples for 24 h;

FIG. 16 shows the tumor growth curves of the colon cancer CT26 tumor bearing mice after treated with different adjuvants combined with inactivated bacteria;

FIG. 17 shows the weight change curves of the colon cancer CT26 tumor bearing mice after treated with different adjuvants combined with inactivated bacteria;

FIG. 18 is a statistical diagram of Cy5.5 signal intensity in CD45 positive cells at tumor sites detected by flow cytometry 24 h after injection of Cy5.5-labeled bacteria (F.S) or Cy5.5-labeled manganese dioxide-biomineralized bacteria (MnO₂@F.S) at the tumor sites;

FIG. 19 shows the distribution of manganese ions in major organs and tumor tissues 24 h after intratumoral injection of different samples in mice;

FIG. 20 shows the statistics of oxygen content inside the tumor at different time points after intratumoral injection of different samples;

FIG. 21 is a statistical diagram of the maturation percentage of DC cells in lymph nodes after tumor bearing mice treated with different samples;

FIG. 22 is a statistical diagram of the percentage of immune cells infiltrating inside the tumor 24 h after the mice received tumor treatment;

FIG. 23 is a statistical diagram of the content of cytokines inside the tumor 24 h after the mice received tumor treatment;

FIG. 24 is a statistical diagram of the percentage of immune cells infiltrating inside the tumor 5 days after the mice received tumor treatment;

FIG. 25 shows the tumor growth curves of the B16-OVA tumor model mice treated with NK cell blocking antibodies and the B16-OVA tumor-bearing normal mice;

FIG. 26 shows the tumor growth curves of the CT26 tumor model of mice treated with T cell blocking antibodies and the CT26-OVA tumor-bearing normal mice;

FIG. 27 shows the tumor growth curves of different tumor models (4T1 breast cancer tumor, B16-OVA melanoma, KPC pancreatic cancer) after treatment;

FIG. 28 shows the tumor growth curves of CT26 tumor model after tumor-bearing mice treated with different manganese-biomineralized bacteria;

FIG. 29 shows the tumor growth curves of 4T1 tumor model after tumor-bearing mice treated with different manganese-biomineralized bacteria;

FIG. 30 shows the tumor growth curves of tumor-bearing mice treated with manganese-biomineralized bacteria prepared with different manganese sulfate/bacteria feed ratios;

FIG. 31 shows the tumor growth curves of the liver cancer New Zealand rabbit model after treatment with manganese-biomineralized bacteria;

FIG. 32 shows the tumor growth curves of the mouse bilateral tumor model after treatment with manganese-biomineralized bacteria;

FIG. 33 is a statistical diagram of the percentage of memory T cells in peripheral blood on the 60th day after the CT26 tumor-bearing mice were cured by manganese-biomineralized bacteria;

FIG. 34 is a graph showing changes in tumor volume in the case of re-inoculation with the tumor on the 60th day after the CT26 tumor-bearing mice were cured by manganese-biomineralized bacteria;

FIG. 35 is an SEM image of manganese-biomineralized inactivated bacteria prepared from manganese sulfate;

FIG. 36 is an SEM image of manganese-biomineralized inactivated bacteria with manganese sulfide adhering to the surface;

FIG. 37 is an SEM image of manganese-biomineralized inactivated bacteria prepared from potassium permanganate;

FIG. 38 is an SEM image of manganese-biomineralized bacteria obtained with different manganese feed ratios.

### DETAILED DESCRIPTION

The embodiments of the invention of the present application will be described below with specific examples. Those skilled in the art can easily understand other advantages and effects of the invention of the present application from the disclosure of the specification.

### Definition of Terms

In the present application, the term “modified” generally refers to some modification compared with the natural state (or wild type), for example, it may be artificial modification. For example, the modification may be a modification by physical means, chemical means, and/or biological means. For example, the modification may be a modification of one aspect or multiple aspects.

In the present application, the term “poorly soluble” is used interchangeably with “slightly soluble” and generally refers to low solubility in a solvent at room temperature (20° C.), for example, the solubility in the solvent is about 0.01 g-1 g/100 g or lower.

In the present application, the term “about” generally refers to a variation within the range of 0.5%-10% above or below the specified numerical value, such as a variation within the range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below the specified numerical value.

In the present application, the term “modified bacterium” not only comprises the modification that the surface of the bacterium body comprises biologically acceptable metal compounds, but also may further include bacteria modified by other methods. For example, the bacterium may be a genetically modified bacterium. For example, the surface of the bacterium body may also comprise other substances. For example, the bacteria may be attenuated strains.

In the present application, the term “composition” generally refers to a composition which, in addition to active ingredients, may also comprise inactive ingredients. For example, the inactive ingredients may include one or more (pharmaceutically acceptable) suitable preparations of carriers, stabilizers, excipients, diluents, solubilizers, surfactants, emulsifiers and/or preservatives. Acceptable ingredients of the composition are generally non-toxic or have minimal toxic side effects on the recipient at the doses and concentrations used.

In the present application, the term “comprising” generally means including, generalizing, containing or encompassing. In some cases, it also means “be” or “consist of”.

In the present application, the term “treatment/treat” generally means: (i) preventing the development of a disease, disorder or condition in a patient who may be susceptible to the disease, disorder and/or condition but has not yet been diagnosed with the disease, disorder or condition; (ii) inhibiting the disease, disorder or condition, i.e., arresting its progression; and (iii) alleviating the disease, disorder or condition, i.e., causing the disease, disorder and/or condition and/or symptoms associated with the disease, disorder and/or condition subside.

In the present application, the term “subject” generally refers to a human or non-human animal, including but not limited to cats, dogs, horses, pigs, cows, sheep, rabbits, mice, rats or monkeys, and the like.

In the present application, the term “tumor” generally refers to neoplastic or malignant cell growth. Tumors in the present application may be benign or malignant. Tumors in the present application may solid or non-solid.

### DETAILED DESCRIPTION OF THE INVENTION

### Modified Bacteria

The present application provides a modified bacterium, a preparation method thereof and an application thereof. In the present application, metal ions are mixed with living bacteria or inactivated bacteria (initially using attenuated Salmonella) and then the pH value of the solution is adjusted to weak alkalinity, which will cause insoluble or poorly soluble metal ion compounds to adhere to the surface of bacteria to form modified bacteria. Through intratumoral injection, the modified bacteria are injected inside the mouse tumor model, which can achieve the effect of inhibiting tumor growth. After further studying the mechanism, it was found that the modified bacteria after in-situ injection caused anti-tumor immune responses in the body, thereby achieving the effect of tumor treatment. When divalent metal ions are placed under alkaline conditions, hydroxides are easily formed and precipitated. As a typical solid-liquid interface, the surface of bacteria can provide a nucleation center for precipitates, further promoting the precipitation of hydroxides. After the hydroxides are precipitated on the surface of bacteria, they are converted into more stable oxides again. With the continuous precipitation and conversion of hydroxides, an oxide adhesion layer is formed on the surface of bacteria, and inactivated bacteria modified with metal compounds are obtained. The deposition reaction described in the present application is that after some metal ion salts are mixed with bacteria, the corresponding anions are introduced, and under appropriate pH conditions, the metal ions and anions form poorly soluble or insoluble metal compounds and adhere to the surface of bacteria; with sufficient stirring, the target product can continuously settle on the surface of bacteria in the liquid, and finally, the bacteria adhered with the metal compound form a uniform and stable suspension.

Further research found that in addition to attenuated Salmonella, this technology can also be used for other types of bacteria, including, for example, Staphylococcus aureus, Escherichia coli and Lactobacillus. Modified bacteria prepared using the above-mentioned bacteria based on the same method can also activate the immune system to obtain good anti-tumor activity.

It is further explored that whether other poorly soluble or insoluble metal compounds other than metal hydroxides or metal oxides can form an adhesion layer on the surface of bacteria and achieve similar immune stimulation results when the solubility is reduced. By trying to synthesize inactivated bacteria adhered with a variety of metal compounds, it is found that some metal compounds, after binding to the surface of bacteria, can also activate immune cells to induce strong anti-tumor immune responses, and may produce immune memory effects to reduce the probability of cancer metastasis and recurrence.

In the present application, the modified bacterium comprises a bacterium body and a poorly soluble or insoluble biologically acceptable metal compound modified on the surface of the bacterium body.

In the present application, the bacterium body may be selected from one or more of Salmonella, Staphylococcus aureus, Escherichia coli, Lactobacillus, attenuated strains of Salmonella, attenuated strains of Staphylococcus aureus, attenuated strains of Escherichia coli and attenuated strains of Lactobacillus.

In the present application, the bacterium body may be a living bacterium or an inactivated bacterium. For example, the bacterium body is an inactivated bacterium, and both the living bacterium and the inactivated bacterium may include attenuated bacteria.

In the present application, the metal compound may be modified on the surface of the bacterium body to form an adhesion layer through deposition.

In the present application, the metal compound has a metal element that may be selected from one or more of zinc, calcium, copper, iron, manganese and magnesium; and a non-metal element that may be selected from one or more of carbonate radical, hydroxide radical, sulfide radical and phosphate radical.

In the present application, the metal compound may be selected from one or more of zinc carbonate, calcium carbonate, copper carbonate, magnesium carbonate; zinc hydroxide, ferric hydroxide, copper hydroxide, manganese hydroxide, magnesium hydroxide, zinc sulfide, copper sulfide, manganese sulfide; zinc phosphate, calcium phosphate, copper phosphate, iron phosphate, magnesium phosphate and manganese phosphate.

The present application further provides a preparation method of the above-mentioned modified bacterium, comprising the following steps:
S1: preparation of a bacterial suspension;
S2: adding a soluble metal salt solution to the bacterial suspension;
step S3: adding an easily soluble aqueous hydroxide solution, aqueous carbonate solution or aqueous phosphate solution to reach a pH of 8-12 (e.g., a pH of about 8, a pH of about 9, a pH of about 10, a pH of about 11 or a pH of about 12), or adding an easily soluble aqueous sulfide solution to react to prepare the modified bacterium. Specifically, in the step S2,
an easily soluble aqueous hydroxide solution can be added to reach a pH of 8-12 (e.g., a pH of about 8, a pH of about 9, a pH of about 10, a pH of about 11 or a pH of about 12), and after the reaction, modified bacteria with metal hydroxide or metal oxide adhered to the surface can be obtained by centrifugation; the easily soluble aqueous hydroxide solution is preferably an aqueous sodium hydroxide solution;
an easily soluble aqueous carbonate solution can be added to reach a pH of 8-12 (e.g., a pH of about 8, a pH of about 9, a pH of about 10, a pH of about 11 or a pH of about 12), and after the reaction, modified bacteria with metal carbonates adhered to the surface can be obtained by centrifugation; the easily soluble aqueous carbonate solution is preferably an aqueous sodium carbonate solution;
an easily soluble aqueous phosphate solution can be added to reach a pH of 8-12 (e.g., a pH of about 8, a pH of about 9, a pH of about 10, a pH of about 11 or a pH of about 12), and after the reaction, modified bacteria with metal phosphates adhered to the surface can be obtained by centrifugation; the easily soluble aqueous phosphate solution is preferably an aqueous sodium phosphate solution;
an easily soluble aqueous sulfide solution can be added, and after the reaction, modified bacteria with metal sulfides adhered to the surface can be obtained by centrifugation; the easily soluble aqueous sulfide solution is preferably an aqueous sodium sulfide solution;
further, in the step S2, a soluble metal salt solution may be added to the bacterial suspension at an amount of 0.2-13.5 mmol of metal ions per 1 billion bacteria. For example, the soluble metal salt solution can be added to the bacterial suspension at an amount of about 0.2 mmol, about 0.5 mmol, about 1.0 mmol, about 2.0 mmol, about 3.0 mmol, about 4.0 mmol, about 5.0 mmol, about 6.0 mmol, about 7.0 mmol, about 8.0 mmol, about 9.0 mmol, about 10.0 mmol, about 11.0 mmol, about 12.0 mmol, about 13.0 mmol, or about 13.5 mmol of metal ions per 1 billion bacteria.

The present application further provides a modified bacterial freeze-dried powder comprising a freeze-drying protective agent and the above-mentioned modified bacterium.

In the present application, the freeze-drying protective agent may be selected from one or more of sucrose, trehalose, inositol, raffinose, inulin, dextran, maltodextrin, maltopolysaccharide, and 2-hydroxypropyl-ß-cyclodextrin.

Further, before the bacterial freeze-dried powder is freeze-dried, the mass/volume fraction of the freeze-drying protective agent in the sample suspension is 0.1-20%, for example, 1-5%. For example, the mass/volume fraction is about 0.1%, about 1%, about 2%, about 3%, about 4%, 5%, about 6%, about 7%, about 8%, about 9%, about 10%, about 11%, about 12%, about 13%, about 14%, about 15%, about 16%, about 17%, about 18%, about 19%, about 20%.

The present application further provides use of the above-mentioned modified bacterium or the above-mentioned modified bacterial freeze-dried powder in the preparation of tumor therapeutics.

In the present application, further provided is a modified bacterium comprising a bacterium body and a poorly soluble or insoluble biologically acceptable metal compound modified on the surface of the bacterium body, wherein the metal compound is modified on the surface of the bacterium body to form an adhesion layer through deposition.

In the present application, further provided is a modified bacterium comprising a bacterium body and a poorly soluble or insoluble biologically acceptable metal compound modified on the surface of the bacterium body, wherein the content of the metal compound on every 1*10⁸ bacteria cells is about 0.1 μg to 600 μg. For example, the content of the metal compound is about 0.1 μg, about 1 μg, about 5 μg, about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 550 μg, or about 600 μg per 1*10⁸ bacteria cells.

In the present application, further provided is a composition comprising the modified bacterium or the manganese-biomineralized bacterium, and optionally a pharmaceutically acceptable carrier, wherein the modified bacterium comprises a bacterium body and a poorly soluble or insoluble biologically acceptable metal compound modified on the surface of the bacterium body, and the pH value of the composition is 0-14. For example, the composition has a pH of about 1, 2, 3, 4, 5, 6, 7, 8, 9, 10, 11, 12, 13, or 14. In the present application, further provided is a preparation method of the modified bacterium or the manganese-biomineralized bacterium, which comprises the following steps: S1: preparation of a bacterial suspension; S2: adding a soluble metal salt solution to the bacterial suspension.

### Manganese-Biomineralized Bacteria

As an example, the present application mixes manganese ions with living bacteria or inactivated bacteria, and it is found that adjusting the pH value of the solution to weak alkalinity will lead to the growth of micron or nanoscale manganese compounds on the surface of bacteria. This process is defined as microbial manganization, and the combination of microbial body and manganese compounds obtained in the process is defined as manganese-biomineralized bacteria. For example, when divalent manganese ions are placed in alkaline conditions, they are easy to form manganese hydroxide and are precipitated, while bacteria are negatively charged under such solution conditions. As a typical solid-liquid interface, the surface of bacteria can provide nucleation centers for precipitates, and especially, some microstructures on the surface of bacteria can further promote the precipitation of manganese hydroxide. When manganese hydroxide is precipitated on the surface of bacteria, it is converted into more stable manganese dioxide again under the action of dissolved oxygen. With the continuous precipitation and conversion of manganese hydroxide, a manganese dioxide adhesion layer is formed on the surface of bacteria to obtain manganese-biomineralized bacteria.

Injecting manganese-biomineralized bacteria with micron or nano-manganese dioxide grown on the surface into solid tumors of various models, it can be found that the growth of tumors injected with manganese-biomineralized bacteria will be extremely effectively inhibited, and some tumors may even be completely ablated; more surprisingly, the biologically manganese-biomineralized inactivated bacteria with manganese dioxide growing on the surface are far more therapeutically effective than manganese dioxide alone, inactivated bacteria alone, and the mixture of manganese dioxide and bacteria, and have no obvious toxic and side effects. Compared with CN201910319344.1 and CN201710795715.4 in the Background, the significant feature is that no tumor-related antigens are introduced in the system of the present application, and the anti-tumor response generated is not limited by the type of tumor antigens, and it is effective against various types of tumors.

Based on the above findings, we further studied the mechanism of manganese-biomineralized bacteria inhibiting tumors: experimental analysis found that such bacteria loaded with manganese dioxide on the surface can be integrally phagocytosed by immune cells in the body. The manganese ions entering the immune cells will stimulate the immune cells to secrete related cytokines, recruit more immune cells to the tumor site, and promote more manganese-biomineralized bacteria to be integrally phagocytosed, further enhancing the immune stimulation efficiency and forming a “positive feedback” process. Compared with a simple mixed system of inactivated bacteria and manganese dioxide, the structural integration of manganese-biomineralized bacteria significantly improves the phagocytosis and stimulation efficiency, allowing immune cells to phagocytose bacteria and manganese dioxide at the same time, so that “dioxide manganese manganese-biomineralized bacteria” have significantly enhanced immune-stimulating effects than “a mixture of manganese dioxide and inactivated bacteria”. Further research shows that after manganese-biomineralized bacteria are injected into tumors, they first activate natural immunity, recruit natural killer cells (NK cells), macrophages, etc. to the tumor site, kill some tumor cells through non-specific immune responses, and expose tumor-associated antigens; subsequently, the manganese ions released from gradual degradation of manganese dioxide can stimulate the STING pathway, and the inactivated bacterium body can stimulate Toll-like receptors (TLRs). The combination of the two mechanisms can more effectively activate antigen-presenting cells such as dendritic cells (DCs), and DCs in turn present tumor antigens to T cells, activating T cell-mediated adaptive immunity (tumor-specific immune response). Tumor-specific T cells migrate throughout the body (including distal tumors), inhibit tumor metastasis, and generate immune memory cells to inhibit tumor recurrence. At the same time, manganese dioxide catalyzes the decomposition of hydrogen peroxide in tumors to produce oxygen, which improves tumor hypoxia, reverses the immunosuppressive tumor microenvironment, and is also helpful to improve the effect of immunotherapy.

Further research found that in addition to attenuated Salmonella, this technology can also be used for other types of bacteria, including, for example, Staphylococcus aureus, Escherichia coli and Lactobacillus with similar effects. manganese-biomineralized bacteria prepared using the above-mentioned bacteria based on the same method can also activate the immune system to obtain good anti-tumor activity. In addition, according to the properties of manganese compounds, manganese-biomineralized bacteria with different manganese compounds growing on the surface can be obtained through different methods. Specifically, two methods, oxidation and reduction, can be used to obtain manganese-biomineralized bacteria with manganese dioxide growing on the surface; or sulfide is added to convert the soluble manganese salt into insoluble manganese sulfide, which grows on the surface of bacteria to obtain manganese-biomineralized bacteria with manganese sulfide growing on the surface.

The beneficial effects of the manganese-biomineralized bacteria of the present application are:
(1) The manganese-biomineralized bacteria provided by the present application can regulate the hypoxic and weakly acidic microenvironment in the tumor. At the same time, they stimulate the host immune system through the STING and Toll-like receptor (TLR) pathways, and successively activate the innate immune response and adaptive immune response, producing a strong and effective anti-tumor immune effect, which is suitable for many types of tumors. At the same time, because this technology can use inactivated bacteria, the manganese-biomineralized inactivated bacteria are safe, well tolerated by mice at high doses and are expected to have a very wide safety window for clinical application.
(2) The manganese-biomineralized bacteria provided by the present application can improve the tumor microenvironment through manganese-containing compounds, can decompose endogenous hydrogen peroxide H₂O₂ in tumor lesions, and significantly improve the hypoxic microenvironment of the tumor site while stimulating the host immune system together with the inactivated bacteria. On the other hand, weakly alkaline manganese-biomineralized bacteria can also appropriately adjust the acidic microenvironment. Since the hypoxic and slightly acidic environment of tumors is an important cause of immunosuppression at the tumor site, manganese-containing compounds can improve tumor hypoxia and neutralize microacidity, which can reverse the immunosuppressive microenvironment within tumors and help the recruited immune cells infiltrate deep into the tumor after immune stimulation with manganese-biomineralized bacteria, thereby significantly enhancing the ability of antigen-presenting cells to present antigens, upregulating the secretion of cytokines, and strengthening subsequent anti-tumor immune responses.
(3) The manganese-biomineralized bacteria significantly improves the phagocytosis and stimulation efficiency through structural integration, allowing immune cells to phagocytose bacteria and manganese dioxide at the same time, so that “dioxide manganese manganese-biomineralized bacteria” have significantly enhanced immune-stimulating effects than “a mixture of manganese dioxide and inactivated bacteria”. Further research shows that after manganese-biomineralized bacteria are injected into tumors, they first activate natural immunity, recruit natural killer cells (NK cells), macrophages, etc. to the tumor site, kill part of tumor cells through non-specific immune responses, and expose tumor-associated antigens; subsequently, the manganese ions released slowly from the manganese-containing compounds can stimulate the STING pathway, and the inactivated bacterium itself can stimulate the Toll-like receptor (TLR) pathway. The combination of the two mechanisms can more effectively activate antigen-presenting cells such as dendritic cells (DCs), and DCs in turn present tumor antigens to T cells, activating T cell-mediated adaptive immunity (tumor-specific immune response). Tumor-specific T cells migrate throughout the body (including distal tumors), inhibit tumor metastasis, and generate immune memory cells to inhibit tumor recurrence. It has an oncolytic effect much stronger than that of manganese-containing compounds alone, inactivated bacteria alone, and the mixture of manganese-containing compounds and bacteria with no obvious toxic and side effects.

In the present application, the manganese-biomineralized bacterium comprises a bacterium body and a poorly soluble or slightly soluble manganese-containing compound adhering to the surface of the bacterium body.

Further, the bacterium body may be a Gram-positive bacterium or a Gram-negative bacterium.

Specifically, the bacterium body may be one or more of Salmonella, Staphylococcus aureus, Escherichia coli, and Lactobacillus.

Further, the poorly soluble or slightly soluble manganese-containing compound may be one or more of manganese hydroxide, manganese dioxide and manganese sulfide, and preferably manganese dioxide.

Specifically, the poorly soluble or slightly soluble manganese-containing compound can be manganese hydroxide, manganese dioxide, manganese sulfide or both manganese hydroxide and manganese dioxide.

Further, the bacterium body may be a living bacterium or an inactivated bacterium, such as an inactivated bacterium, and both the living bacterium and the inactivated bacterium include attenuated bacteria.

The present application further provides a manganese-biomineralized bacterial freeze-dried powder comprising any of the above-mentioned manganese-biomineralized bacteria and a freeze-drying protective agent; the freeze-drying protective agent is at least one of sucrose, mannose, a-D-mannopyranose, trehalose, inositol, raffinose, inulin, dextran, maltodextrin, maltopolysaccharide, sucrose octasulfate, heparin and 2-hydroxypropyl-β-cyclodextrin.

Further, the mass/volume fraction of the freeze-drying protective agent in the bacterial freeze-dried powder prior to freeze-drying may be 0.1%-20%.

The present application further provides a preparation method of manganese-biomineralized bacteria, comprising the following steps:

S1: preparation of a bacterial suspension;

S2: adding a manganese salt solution, adjusting the pH to weak alkalinity of 8-12 (pH is 8-12, for example, the pH range is 10-11), reacting for 1-2 h, and centrifuging to prepare manganese-biomineralized bacteria with manganese hydroxide and/or manganese dioxide coated on the surface.

Or S2′: adding an appropriate amount of a manganese salt solution to the bacterial suspension obtained in step S1, adding a soluble sulfide salt solution, reacting for 1-2 h, and centrifuging to prepare manganese-biomineralized bacteria with manganese sulfide coated on the surface.

Further, the bacterium is a living bacterium or an inactivated bacterium, such as an inactivated bacterium.

Further, the bacterium may be a Gram-positive bacterium or a Gram-negative bacterium.

Specifically, pH adjustment can be achieved by adding a sodium oxide solution. Soluble sulfide salt solutions include sodium sulfide solutions, potassium sulfide solutions, ammonium sulfide solutions, etc.

Further, 0.2 μmol-13.5 mmol of a manganese salt can be added per 1 billion

bacteria. For example, about 0.2 μmol, about 0.5 μmol, about 1.0 μmol, about 2.0 μmol, about 3.0 μmol, about 4.0 μmol, about 5.0 μmol, about 6.0 μmol, about 7.0 μmol, about 8.0 μmol, about 9.0 μmol, 0.02 mmol, about 0.05 mmol, about 0.1 mmol, about 0.2 mmol, about 0.5 mmol, about 1.0 mmol, about 2.0 mmol, about 3.0 mmol, about 4.0 mmol, about 5.0 mmol, about 6.0 mmol, about 7.0 mmol, about 8.0 mmol, about 9.0 mmol, about 10.0 mmol, about 11.0 mmol, about 12.0 mmol, about 13.0 mmol, or about 13.5 mmol of a manganese salt is added per 1 billion bacteria.

In the present application, further provided is a preparation method of manganese-biomineralized bacteria, comprising the following steps:
SS1: preparation of a bacterial suspension;
SS2: adding a permanganate solution to react with stirring and centrifuging to prepare the manganese-biomineralized bacteria.

Further, the bacterium is a living bacterium or an inactivated bacterium, such as an inactivated bacterium.

Further, the bacterium is a Gram-positive bacterium or a Gram-negative bacterium. Further, 0.2-3 μmol of a permanganese salt is added per 1 billion bacteria.

The present application further provides use of any of the above-mentioned manganese-biomineralized bacteria in the preparation of tumor immunotherapeutics.

The present application further provides use of the freeze-dried powder prepared by any of the above-mentioned manganese-biomineralized bacteria in the preparation of tumor immunotherapeutics.

### Bacterium Body

In the present application, the bacterium body may be a Gram-positive bacterium or a Gram-negative bacterium.

In the present application, the bacterium body may be selected from one or more of coccus, bacillus, and spirillum. For example, the coccus may be selected from one or more of Staphylococcus aureus, Micrococcus urea, Diplococcus pneumoniae, Streptococcus pneumoniae, Diplococcus meningitidis, Streptococcus pyogenes, Streptococcus pneumoniae, Streptococcus lactis, Staphylococcus aureus, Staphylococcus albus, and Staphylococcus citreus. For example, the bacillus may be selected from one or more of Escherichia coli, Salmonella, Yersinia pestis, Bacillus dysenteriae suis, Pasteurella multocida, Corynebacterium diphtheriae, Mycobacterium tuberculosis Bacillus, Lactobacillus bifidum, Bacillus aceticus, and Corynebacterium. For example, the spirillum may be selected from one or more of Helicobacter pylori, Vibrio comma and Vibrio cholerae.

In the present application, the bacterium body may be selected from one or more of Salmonella, Staphylococcus aureus, Escherichia coli, Lactobacillus, attenuated strains of Salmonella, attenuated strains of Staphylococcus aureus, attenuated strains of Escherichia coliand attenuated strains of Lactobacillus.

In the present application, the bacterium body may be a wild-type strain, a genetically engineered strain, and/or an attenuated strain.

In the present application, the bacterium body may be a living bacterium or an inactivated bacterium. For example, the living bacterium or inactivated bacterium may include attenuated bacteria.

### The Adhesion Methods of Metal Compounds

In the present application, the metal compound (for example, a manganese-containing compound) can be modified on the surface of the bacterium body to form an adhesion layer by physical binding. For example, the physical binding may include electrostatic adsorption. For example, the physical binding may include partial embedded.

In the present application, the metal compound (for example, a manganese-containing compound) can be modified on the surface of the bacterium body to form an adhesion layer by chemical binding. For example, the chemical binding may include coupling. For example, the chemical binding may include formation of chemical bonds. For example, the chemical binding may include binding by complexation.

In the present application, the metal compound (for example, a manganese-containing compound) may be modified on the surface of the bacterium body to form an adhesion layer through deposition.

In the present application, the metal compound (for example, a manganese-containing compound) can be modified on the surface of the bacterium body to form an adhesion layer through biomineralization. As used herein, the biomineralization generally means that metal ions bind to biomacromolecules on the cell membrane or biomacromolecules on the cell wall of bacteria to provide mineralization sites, pH is adjusted or other salts are introduced, the metal ions generate metal compounds at the mineralization sites, the metal compounds continue to grow and accumulate and bind to the surface of bacteria. For example, binding sites for biomacromolecules and metal compounds can be formed through the biomineralization, and the volume of the metal compound is further increased by deposition or biomineralization.

In the present application, the coverage of the metal compound on the surface of the bacterium may be about 0.1%-99.9%. At this coverage, bacteria are able to come into contact with the physiological environment and stimulate the immune system. For example, the coverage of the metal compound on the surface of the bacterium is adjusted by adjusting feed ratio, reaction temperature and/or reaction time.

In the present application, the coverage of the metal compound on the surface of bacteria is (bacterial surface area - area of modified bacteria in direct contact with the outside world)/bacterial surface area.

### Modified Bacterial Freeze-Dried Powder

In the present application, the modified bacterial freeze-dried powder (e.g., manganese-biomineralized bacterial freeze-dried powder) may comprise modified bacteria (e.g., manganese-biomineralized bacteria), and additives.

In the present application, the additive may include a freeze-drying protective agent. For example, the freeze-drying protective agent my be selected from one or more of sugars/polyols, polymers, anhydrous solvents, surfactants, amino acids, salts and amines. For example, the freeze-drying protective agent may be selected from one or more of sucrose, mannose, a-D-mannopyranose, trehalose, inositol, raffinose, inulin, dextran, maltodextrin, maltopolysaccharide, sucrose octasulfate, heparin and 2-hydroxypropyl-β-cyclodextrin.

In the present application, the mass fraction of the freeze-drying protective agent in the modified bacterial freeze-dried powder is 0.1-99%. For example, the mass fraction of the freeze-drying protective agent is about 1%, about 2%, about 3%, about 5%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%. For example, the mass fraction of the freeze-drying protective agent in the modified bacterial freeze-dried powder is 0.1-20%.

In the present application, the additives may include excipients. For example, the excipient may be selected from one or more of binders, fillers, disintegrants, lubricants, wine, vinegar, decoctions, etc., ointment bases, cream bases, preservatives, antioxidants, flavoring agents, fragrances, co-solvents, emulsifiers, solubilizers, osmotic pressure regulators and colorants.

In the present application, the mass fraction of the additive in the modified bacterial freeze-dried powder is 0.1-99%. For example, the mass fraction of the additive is about 1%, about 2%, about 3%, about 5%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%.

Without wishing to be limited by any theory, the following examples are only for illustrating various technical solutions of the invention of the present application, and are not intended to limit the scope of the invention of the present application.

### EXAMPLES

### Material Sources

The sources of the strains in the examples of the present application are as shown in the following table:

| | | | Article No./reference |
|---|---|---|---|
| Strain name | Source | How to obtain | document |
| Salmonella | ATCC | Purchased | ATCC14028 |
| Attenuated | Xiangya | Donated | ppGpp-dependent Stationary |
| Salmonella | Hospital Central | | Phase Induction of Genes on |
| | South University | | Salmonella Pathogenicity |
| | | | Island, DOI |
| | | | 10.1074/jbc.M313491200; |
| | | | Xuan Yi, Hailin Zhou, Yu |
| | | | Chao, Saisai Xiong, Jing |
| | | | Zhong, Zhifang Chai, Kai |
| | | | Yang*, and Zhuang Liu*. |
| | | | “Bacteria-triggered tumor- |
| | | | specific thrombosis to enable |
| | | | potent photothermal |
| | | | immunotherapy of cancer” Sci. |
| | | | Adv., 6(33): eaba3546 (2020) |
| VNP20009 | ATCC | Purchased | ATCC 202165 |
| Staphylococcus | Affiliated | Donated | Chenya Wang, Yuanpeng |
| aureus | Hospital of | | Xiao, Wenwen Zhu, Jiacheng |
| | Soochow | | Chu, Jun Xu, He Zhao, |
| | University | | Fengyun Shen, Rui Peng*, |
| | | | Zhuang Liu *.” Photosensitizer- |
| | | | Modified MnO2 Nanoparticles |
| | | | to Enhance Photodynamic |
| | | | Treatment of Abscesses and |
| | | | Boost Immune Protection for |
| | | | Treated Mice” Small, 2000589 |
| | | | (2020) |
| Escherichia coli | Affiliated | Donated | Qi Zhuang, Jun Xu, Dashi |
| | Hospital of | | Deng, Ting Chao, Junyan Li, |
| | Soochow | | Rui Zhang, Rui Peng*, Zhuang |
| | University | | Liu *.” Bacteria-derived |
| | | | Membrane Vesicles to |
| | | | Advance Targeted |
| | | | Photothermal Tumor Ablation” |
| | | | Biomaterials, 268, 120550 |
| | | | (2020) |
| Lactobacilli | Zhengzhou | Purchased | Lactobacillus rhamnosus |
| | Baiyibao | | |
| | Biological | | |
| | Technology Co., | | |
| | Ltd. | | |

The sources of the cells in the examples of the present application are as shown in the following table:

| Name in the | | | |
|---|---|---|---|
| specification | Specific name | Article No. | Source |
| Cells with | Dual IRF and IFN-β reporter 293 | 293d-mstg | InvivoGen |
| reporter genes | cells expressing murine STING | | |
| regulated by | | | |
| activation of | | | |
| STING pathway | | | |
| Cells with | HEK-Blue ™ mTLR4 Cells | hkb-mtlr4 | InvivoGen |
| reporter genes | | | |
| regulated by | | | |
| activation of | | | |
| TLR4 pathway | | | |
| DC2.4 | / | / | Millipore |
| CT26 | CSTR: 19375.09.3101MOUTCM37 | TCM37 | Cell Bank of |
| | | | Shanghai |
| | | | Institutes of |
| | | | Biological |
| | | | Sciences, Chinese |
| | | | Academy of |
| | | | Sciences |

$$The tumor inhibition rate calculation formula used in the examples is : tumor inhibition rate = [ ( 1 - the average tumor volume at the end of treatment in a treatment group - the average tumor volume at the start of treatment in the treatment group ) / ( the average tumor volume at the end of treatment in the control group - average tumor volume at the start of treatment in the control group ) ] \times 100 \%$$

### Example A

### Preparation and Basic Morphology Characterization of Bacteria Modified with Different Metal Compounds

### Example A1

### Preparation of Bacteria Modified by Zinc Carbonate on the Surface

S1: An aqueous zinc sulfate solution was prepared;

S2: Attenuated Salmonella (hereinafter referred to as F.S) was amplified and cultured, centrifuged to obtain bacterial cells, which were cleaned to remove the residual culture medium and other substances in the bacterial cells; then, paraformaldehyde was added to the bacterial cells to inactivate the bacteria and fix the morphology, the fixed inactivated bacteria were collected by centrifugation, and the bacterial cells were washed twice with sterile normal saline, and the inactivated bacterial cells were finally re-suspended in normal saline to obtain an inactivated bacterial suspension with a concentration of 24 MCF; it was detected and confirmed that the bacterial suspension had no living bacteria and could be stored at a low temperature for later use;

S3: 1 mL of a zinc sulfate solution with a concentration of 10 mM and an appropriate amount of normal saline were added to the inactivated bacterial suspension (the turbidity of the original bacterial suspension was 24 MCF, approximately 7.2 billion bacteria/mL, and the volume was 1 mL), stirred briefly at room temperature, and then a sodium carbonate solution was added to make the pH be 8-12; the mixture was further stirred for no less than 1 h, centrifuged to collect the precipitate, which was washed twice with sterile normal saline, and finally re-suspended in sterile normal saline and stored at a low temperature, and the product was ZnCO₃@F.S.

In the experiment, we tried to add bacterial suspensions and metal ion salt solutions with different original concentrations and volume ratios to explore the range of feed ratios that can facilitate the formation of bacteria modified with metal compounds. With the total amount of bacteria in the reaction system fixed, through adjusting the loading amount of the salts of metal ions, the feed ratio when the product was a stable suspension was explored. It was found that when the amount of bacteria was 1 billion and the concentration of metal ions exceeded 13.5 mmol, no matter how the pH, concentration and reaction time of the reaction system were adjusted, the products were all unsuspendable precipitates and could not be used in subsequent applications. When the content of metal ion salts participating in the reaction was low, it did not affect the re-suspension of the product. In the experiment, the lowest feed ratio tried was 0.2 μmol of metal ions per 1 billion bacteria.

### Example A2

### Preparation of Bacteria with Surface Modified by Ferric Hydroxide or Copper Hydroxide

The soluble ion salt solution of iron and the inactivated bacterial suspension were prepared according to the method of S1-S2 in Example A1; the difference is:
S3: A 10 mM ferric chloride or copper sulfate solution and an appropriate amount of normal saline were added to the inactivated bacterial suspension (the turbidity of the original bacterial suspension was 24 MCF, approximately 7.2 billion bacteria/mL, and the loading volume here was 1 mL), stirred briefly at room temperature, and then a sodium hydroxide solution or ammonia was added to adjust the pH to 8-12; the mixture was further stirred for no less than 1 h, centrifuged to collect the precipitate, which was washed twice with sterile normal saline, and finally re-suspended in normal saline and stored at a low temperature, and the products were Fe(OH)3@F.S, Cu(OH)₂@F.S.

### Example A3

### Preparation of Modified Bacteria with Manganese Sulfide, Zinc Sulfide and Copper Sulfide Adhering to the Surface

S1: Aqueous solutions of manganese chloride, copper sulfate, and zinc sulfate with a concentration of 10 mM were prepared;
S2: Attenuated Salmonella was amplified and cultured, and then cleaned to obtain culture medium-free bacterial cells. Without treatment with formaldehyde, the bacterial cells were directly re-suspended in normal saline to obtain a bacterial suspension of living bacteria;
S3: A 10 mM manganese chloride, copper sulfate or zinc sulfate solution respectively and an appropriate amount of normal saline were added to the bacterial suspension (the turbidity of the original bacterial suspension was 24 MCF, approximately 7.2 billion bacteria/mL, and the loading volume here was 1 mL), stirred at room temperature, and then a sodium sulfide solution was added; the mixture was further stirred, centrifuged to collect the precipitate, which was washed twice with sterile normal saline, and finally re-suspended in normal saline to obtain MnS@F.S, CuS@F.S, ZnS@F.S respectively, which were stored at a low temperature.

### Example A4

### Basic Characterization of Modified Bacteria

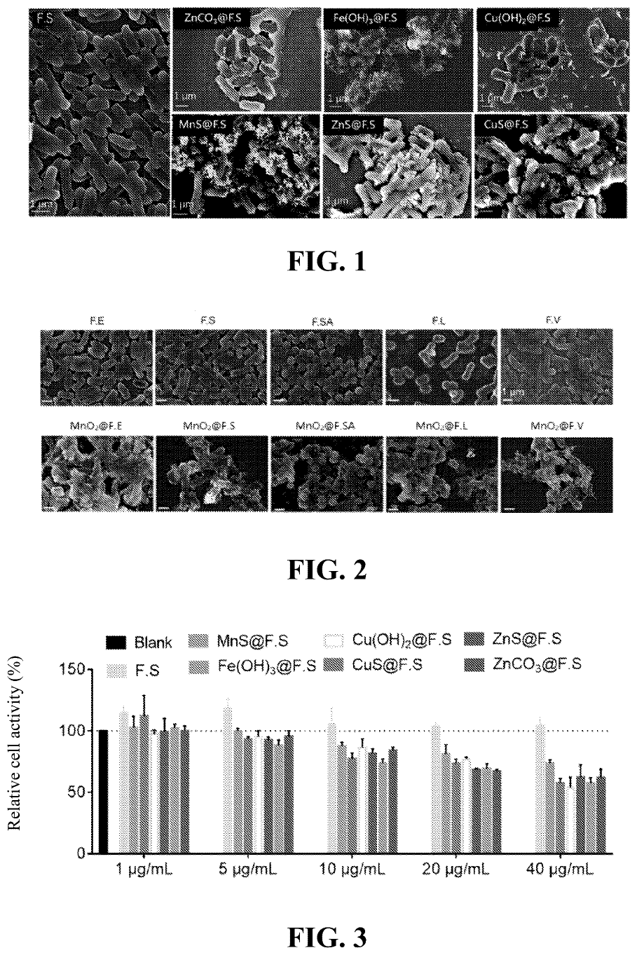

SEM samples were prepared from the products in Examples A1 to A3, and the surface morphology of the bacteria modified with different metal compounds was characterized using a scanning electron microscope (SEM). The results are shown in FIG. 1. The pictures labeled with ZnCO₃@F.S, Fe(OH)₃@F.S, Cu(OH)₂@F.S, MnS@F.S, ZnS@F.S, and CuS@F.S respectively represent SEM images of Salmonella modified with zinc carbonate, ferric hydroxide, copper hydroxide, manganese sulfide, zinc sulfide, and copper sulfide on the surface. The results show that there were a large number of solids adhering to the surface of all the bacteria modified with the metal compounds, and there were no excessive solid substances remaining in the environment. Moreover, according to the different crystal forms of the metal compounds themselves, the morphology of the surface of bacteria after being adhered with different metal compounds also changed, indicating that during the preparation process, the metal compounds adhered to the surface of bacteria to form a covering layer.

### Example A5

### Preparation of Other Modified Bacteria

S1: An aqueous solution of manganese chloride with a concentration of 10 mM was prepared;
S2: Amplification and culture of bacteria, where 50 uL of a frozen bacterial suspension was inoculated into 6 mL of fresh meat extract peptone (LB) medium, the cell culture tube was obliquely put into a constant-temperature shaker at 37° C. for activation at a shaking frequency of 220 r/min for 12 h; then the activated bacterial suspension was diluted and coated on an LB solid medium plate, placed in a digital incubator to culture at 37° C. for 12 h. Single colonies in good condition were picked and inoculated into 5 mL of fresh LB broth medium, and placed on a constant temperature shaker at 37° C. to culture overnight to obtain a bacterial suspension;
S3: 50 μL of the bacterial suspension obtained in S2 was added to 20 mL of fresh LB liquid medium, the cell culture tube was obliquely put into a constant-temperature shaker at 37° C. for activation at a shaking frequency of 220 r/min for 12 h; the bacterial cells were collected by centrifugation, re-suspended in sterile normal saline, and centrifuged again; then 5 mL of 4% paraformaldehyde was added to fix for 24 h, the fixed inactivated bacteria were collected by centrifugation, the bacterial cells were washed twice with sterile normal saline, and finally, the inactivated bacteria were re-suspended in 5-8 mL of normal saline so that the final concentration of the bacterial suspension was 24 MCF, that is, the absorbance of the bacterial suspension at a wavelength of 600 nm was 3; an inactivated bacterial suspension was obtained, which could be stored at 4° C.; 50 μL of the bacterial suspension was taken and spread on solid culture plate, and the absence of living bacteria in the bacterial suspension was confirmed;
S4: A 10 mM manganese chloride solution and an appropriate amount of normal saline were added to the inactivated bacterial suspension and stirred for 15 min, and then a NaOH solution was added to adjust the pH of the solution to 10.0-11.0; the mixture was further stirred for 1 h, centrifuged to collect the precipitate, which was washed twice with sterile normal saline, and finally re-suspended in normal saline and stored at 4° C.

Scanning electron microscopy (SEM) samples of the synthesized manganese-biomineralized inactivated bacteria and non-manganese-biomineralized inactivated bacteria were respectively prepared, and their appearance and morphology were characterized by SEM. The obtained SEM images are shown in FIG. 2, where F.E represents fixed inactivated Escherichia coli, F.SA represents fixed inactivated Staphylococcus aureus, F.S represents fixed inactivated Salmonella, F.L represents fixed inactivated Lactobacillus, F.V represents fixed inactivated Salmonella VNP20009, and the manganese-biomineralized bacteria are indicated by the MnO₂@prefix. As can be seen from FIG. 2, the fixed inactivated bacteria maintained the appearance characteristics of the original bacteria, the surface of the manganese-biomineralized inactivated bacteria was adhered by a layer of rough precipitates, and the bacteria themselves maintained their original shape and size, indicating that the manganization process does not destroy the morphology of the bacteria themselves, but only has a layer of substances adhering to their surface.

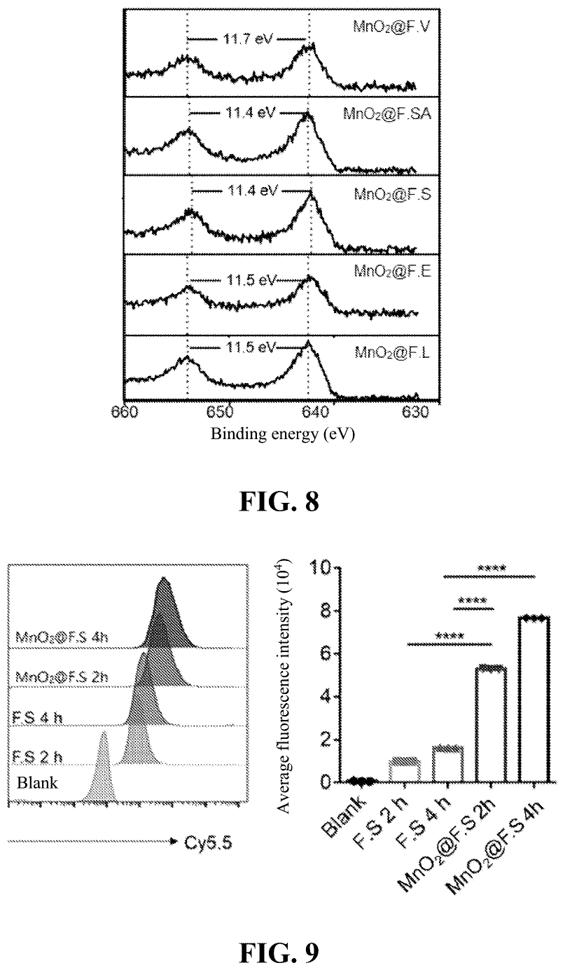

Subsequently, the substances adhering to the surface of bacteria were characterized, and X-ray photoelectron spectroscopy (XPS) technology was used to analyze the elements on the surface of bacteria and their valence states. The results are shown in FIG. 8. The XPS spectra of surface of different bacteria have two characteristic peaks, respectively located at around 654.2 eV and 642.4 eV, corresponding to the 2p1/2 and 2p3/2 spin orbit energy levels of tetravalent manganese ions (Mn⁴⁺) respectively. In conjunction with the element types in the reaction system and the XPS results, it shows that it is manganese dioxide rather than other forms of the element manganese on the surface of the manganese-biomineralized inactivated bacteria.

### Example B

### Cell Experiment

Example B1: Relative cell activity of CT26 cells (mouse colon cancer cell line) after incubation with different samples CT26 cells were inoculated in a 96-well plate at a density of 10⁴ cells/well and cultured in a cell incubator at 37° C. overnight. When the cells were adherent and in good condition, the cells were co-incubated with different samples prepared in Examples A1-A3 (wherein the bacteria were inactivated attenuated Salmonella) of different concentrations at 37° C. For bacteria modified with different metal compounds, the corresponding metal compound concentrations were 1 μg/mL, 5 μg/mL, 10 μg/mL, 20 μg/mL, and 40 μg/mL. In the group of unmodified bacteria, the total amount of bacteria was the same as the total amount of modified bacteria. After incubation for 12 h, the culture medium containing the sample was removed, the excess sample was washed away with PBS, and the relative activity of the cells was detected using the MTT assay. The results are shown in FIG. 3 (the data in each concentration from left to right are: F.S, MnS@F.S, CuS@F.S, Cu(OH)₂@F.S, ZnS@F.S, Fe(OH)3@F.S, ZnCO₃@F.S). At low concentrations, the modified bacteria did not show high cytotoxicity, and the cells co-incubated with the modified bacteria still had more than 80% activity. As the sample concentration increased, the modified bacteria gradually showed effect on the relative activity of cells: when the concentration reached 40 μg/mL, the cell activity was still greater than 50%. It shows that the safety of modified bacteria is related to the concentration used, and it has high safety within a certain concentration range.

### Example B2

### Experiments on Different Samples Stimulating the Maturation of Bone Marrow-Derived Dendritic Cells

DC cells are efficient antigen-presenting cells in the body. They differentiate into mature DC cells after being stimulated by certain factors or ingesting antigens. Mature DC cells can effectively activate naive T cells, induce the generation of cytotoxic T lymphocytes, and secrete tumor necrosis factor α (TNF-α), etc., so that they play a key role in the anti-tumor immune response. Therefore, samples that can effectively stimulate DC cell maturation can enhance the body's anti-tumor immune response.

Bone marrow-derived stem cells were extracted from the bone marrow of C57BL/6 mice, and colony-stimulating factor (GM-CSF) was added to promote the differentiation of stem cells into bone marrow-derived dendritic cells (BMDCs). BMDCs were co-incubated with different modified bacterial samples (the amount of bacteria in each sample was 3.6*10⁷; the corresponding bare metal compound content was 1.5-2.7 μg, in the remaining groups without bacteria, the amount of the same metal compound was the same as in the group of bacteria modified with the metal compound; for example, the ZnS and ZnS@F.S groups contained equal amounts of ZnS), and the maturation percentages of BMDCs in different groups were detected after 12 h.

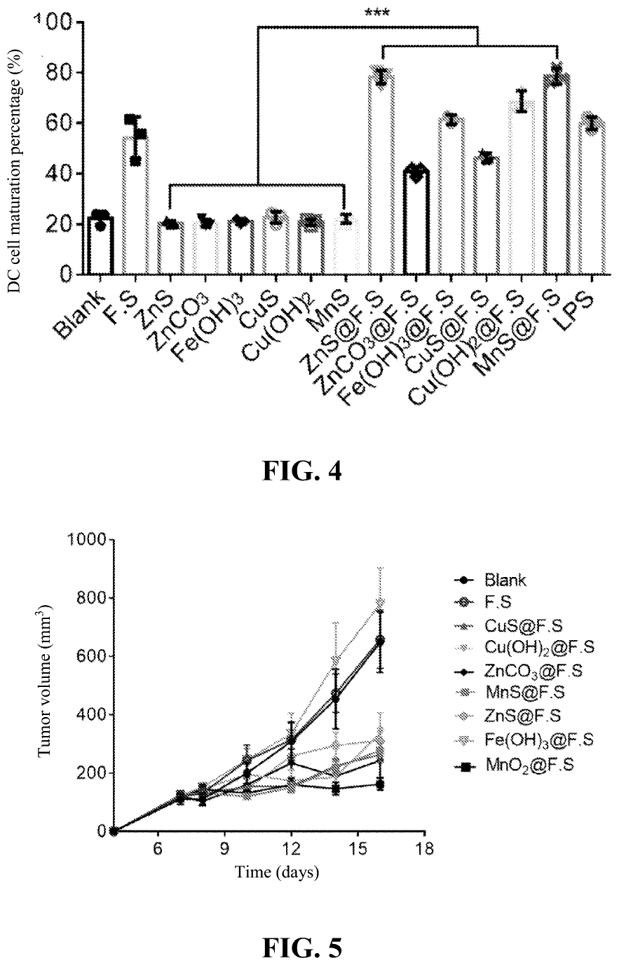

The statistical results of the maturation percentage of BMDCs are shown in FIG. 4. The various experimental groups represent that different substances are added to the cell culture medium and co-incubated with BMDCs. The specific substances are as follows:

blank: blank control group;

F.S: unmodified inactivated bacteria; ZnS: zinc sulfide mixed suspension; ZnCO3: zinc carbonate mixed suspension; Fe(OH)3: ferric hydroxide colloid; CuS: copper sulfide mixed suspension; Cu(OH)2: copper hydroxide mixed suspension; MnS: manganese sulfide mixed suspension;

ZnS@F.S: zinc sulfide-modified Salmonella suspension; ZnCO3@F.S: zinc carbonate-modified Salmonella suspension; Fe(OH)3@F.S: ferric hydroxide-modified Salmonella suspension; CuS@F.S: copper sulfide-modified Salmonella suspension; Cu(OH)2@F.S: copper hydroxide-modified Salmonella suspension; MnS@F.S: manganese sulfide-modified Salmonella suspension.

As can be seen from FIG. 4, the modified bacteria stimulated the maturation percentage of BMDCs to be more than 40%, while the maturation percentage of cells after co-incubation of metal compounds alone with BMDCs did not exceed 20%. Based on the statistics of DC cells maturation percentage, it can be concluded that the modified bacteria can better stimulate the maturation of BMDCs, the results of most groups were significantly different from those of the blank control group (blank), and some experimental groups even obtained better results than the positive control group (2 μg/mL lipopolysaccharide, LPS), indicating that the modified bacteria of the present application have excellent ability to stimulate DC cell maturation. LPS is lipopolysaccharide, a strong activator of immune cells (including B cells, monocytes, macrophages and other LPS-reactive cells). Immature DC cells tend to mature under the stimulation of such immunogenic stimuli. It is a commonly used positive control substance in in vitro DC cell maturation experiments.

### Example C

### Animal Experiment

Example C1: Experiment on treatment of mouse colon cancer tumor model using bacteria modified with different metal compounds

Colon cancer tumor cells were inoculated on the back of BALB/c mice to establish a tumor model. When the tumor grew to a size of about 120 mm³, they were randomly divided into groups of 6 mice each. Different groups of mice received bacteria modified with different metal compounds. A single treatment was delivered via intratumoral injection. Among them, the dosage of bacteria among the control groups was constant, which was 1.8*1010 bacteria/kg body weight. According to the difference in the binding efficiency of each metal compound with the bacteria, the dosage of the metal compound was 0.75 mg/kg-1.35 mg/kg body weight, and the metal ion concentration was 300 μg/mL-1.08 mg/ml. During this period, the changes in tumor volume were recorded and the tumor growth curve was plotted. The results are shown in FIG. 5. At the same time, the survival rate of the mice was recorded and the tumor inhibition rate was calculated. The survival rate statistics and tumor inhibition rate calculation results are shown in Table 1.

TABLE 1
Survival rate of mice after tumor treatment with bacteria
modified with different metal compounds, and tumor inhibition
rate on the 19th day after inoculation

| | Survival rate (%) | | | | | | Tumor |
|---|---|---|---|---|---|---|---|
| | 10 | 15 | 17 | 19 | 21 | 25 | inhibition |
| Group | days | days | days | days | days | days | rate (%) |
| Control group | 100 | 100 | 83.3 | 0 | 0 | 0 | |
| F•S | 100 | 100 | 83.3 | 0 | 0 | 0 | 2.42 |
| MnO₂@F•S | 100 | 100 | 100 | 83.3 | 66.7 | 66.7 | 90.62 |
| CuS@F•S | 100 | 100 | 100 | 83.3 | 66.7 | 33.3 | 74.06 |
| MnS@F•S | 100 | 100 | 100 | 66.7 | 66.7 | 50 | 71.85 |
| ZnS@F•S | 100 | 100 | 83.3 | 83.3 | 50 | 33.3 | 64.12 |
| Fe(OH)₃@F•S | 100 | 100 | 66.7 | 0 | 0 | 0 | 1.04 |
| Cu(OH)₂@F•S | 100 | 100 | 100 | 83.3 | 83.3 | 50 | 60.21 |
| ZnCO₃@F•S | 100 | 100 | 100 | 100 | 83.3 | 66.7 | 77.20 |

The results in FIG. 5 and Table 1 show that compared with treatment with inactivated bacteria alone, the tumor inhibition rate of bacteria modified by different metal compounds for treatment of mouse tumors was significantly improved, indicating that bacteria modified with different metal compounds are more effective and have better therapeutic effects than bacteria alone. Bacteria modified with certain metal compounds can inhibit the growth of tumors. After receiving treatment with bacteria adhered with different metal compounds, the increase in tumor volume of mice significantly slowed down, and the survival rate was significantly increased, with a maximum survival rate of 66.7%, and tumor inhibition rate was basically above 60%. In summary, it is proved that most of the bacteria modified with metal compounds in the present application can achieve therapeutic effects on tumors, while bacteria modified with the hydroxide of Fe (Fe(OH)3@F.S) did not show obvious tumor inhibitory effects. The possible reason is that it is different in dosage for different metal compounds to exert their therapeutic effect, and the effective dosage of Fe(OH)3@F.S was not found in one experiment.

### Example D

### Study on the Immunostimulatory Mechanism of Modified Bacteria

### Example D1

### Proving the Activation of STING Pathway by Modified Bacteria at the Cellular Level

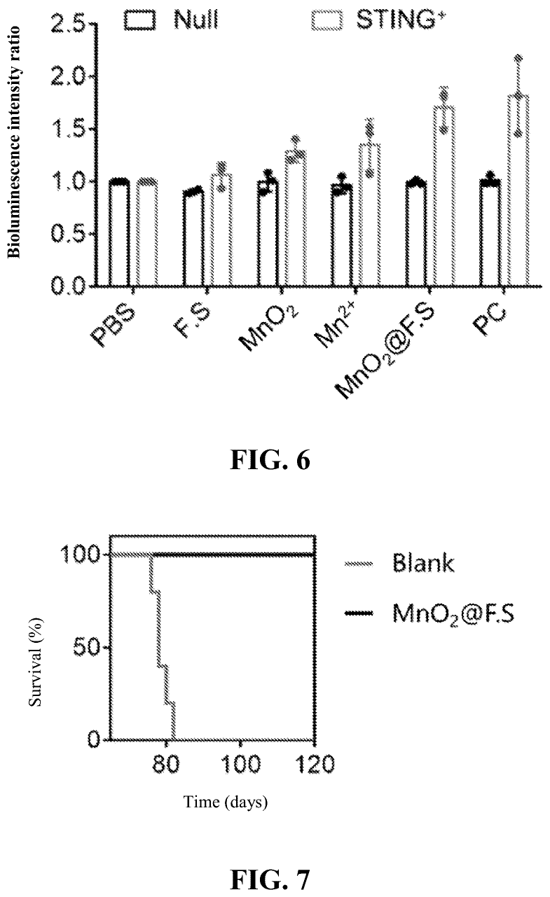

The modified bacteria described in the present application have multi-pathway stimulating effects. This example is designed using Salmonella coated with manganese compounds (manganese dioxide) as an example. Other metal ions have their corresponding immune stimulation mechanisms. In this example, cells containing a reporter gene regulated by STING pathway activation (STING) were used. When the STING pathway of these cells is stimulated and activated, it will activate the expression of the reporter gene luciferase, which can catalyze the luciferase substrate in the culture medium to emit bioluminescence signals. The higher the degree of STING activation, the stronger the bioluminescence signals. The cells were co-incubated with manganese dioxide-modified bacteria (MnO₂@F.S), manganese dioxide (MnO₂), Fixed Salmonella (F.S), manganese chloride (Mn²⁺), and positive control (PC). After 24 h, the bioluminescence signal intensity was detected after adding luciferase substrate, and then the bioluminescence signal intensity of each of the groups was compared with that of the PBS group. Null cells are cells that do not express the STING pathway, so they cannot induce luciferase expression by activating the STING pathway. Incubation of these cells with different samples proves that the samples and reagents in the experiment themselves cannot interfere with the bioluminescence signal or induce luciferase expression. The statistical diagram of bioluminescence signal intensity ratio is shown in FIG. 6. Compared with the control group, the bioluminescence signal intensity ratio of cells co-incubated with modified bacteria was higher, indicating that the STING pathway of cells in this group was significantly activated; compared with the positive control (PC), their bioluminescence signal intensity ratios were close, indicating that their STING activation levels are similar, that is, the modified bacteria can effectively activate the STING pathway.

STING refers to stimulator of interferon genes, which is mainly expressed on the outer membrane of rough endoplasmic reticulum, mitochondria and microsomes of human macrophages, T lymphocytes, dendritic cells, etc. STING plays an important pivotal role in the innate immune response triggered by viral, bacterial and parasitic infections, the body's tumor immune process and the cell autophagy process; it regulates protein synthesis and IFN expression through its own phosphorylation, ubiquitination and dimerization, and plays a key role in many immune processes in the body. STING is an important regulatory target in the body's anti-tumor immunity. Tumor cell proliferation can activate STING in antigen-presenting cells, thereby activating the T cell-mediated adaptive immune process to exert anti-tumor effects. Therefore, the modified bacteria of the present application can stimulate STING pathway activation, indicating that they have the potential for anti-tumor immunotherapy.

### Example D2

### Proving Activation of TLR4 Pathway by Modified Bacteria Through Cell Experiments

In this example, cells (TLR4⁺, TLR4 positive cells) containing reporter genes regulated by TLR4 pathway activation were used. After their TLR4 is stimulated and activated, the expression of reporter gene luciferase will be activated, which can catalyze luciferase substrate to generate bioluminescence signals. The cells were incubated with different samples (n=3). At the end of the incubation, luciferase substrate was added, and the bioluminescence signal intensity was then detected. Then, the bioluminescence signal intensity of each of the groups was compared with that of the PBS group, which can be used to determine the degree of TLR4 activation. The higher the ratio, the greater the degree of TLR4 activation. TLR4-negative cells do not express TLR4, so they will not be stimulated to express luciferase and will not produce bioluminescence after luciferin substrate is added. This group of cells were used to prove that all reagents or samples used in the experiment cannot interfere bioluminescence signals or induce luciferase expression.

The bioluminescence signal intensity ratios of three parallel samples in different groups are shown in Table 2. The positive control group was 3 μg/mL MPLA co-incubated with cells, which had an obvious stimulating effect on the TLR4 pathway. The modified bacteria (MnO₂@F.S) can significantly stimulate TLR4 compared with the blank control group (Blank), with a fluorescence intensity ratio greater than 1; compared with simple bacteria (F.S), the modified bacteria had stronger fluorescence intensity, indicating that the modified bacteria can better stimulate the TLR4 pathway. It shows that the modified bacteria of the present application have an effect similar to TLR4 agonists.

TLR4 is a Toll-like receptor. The activation of TLR4 can promote DCs to secrete relevant interleukins, thereby enhancing the Th1 immune response, which is beneficial to anti-tumor immunotherapy.

TABLE 2
Statistics of bioluminescence intensity ratio after adding luciferase
substrate to cell culture media of different groups

| | TLR4 negative cells | | | TLR4 positive cells | | |
|---|---|---|---|---|---|---|
| Blank | 1.000 | 1.000 | 1.000 | 1.000 | 1.000 | 1.000 |
| F•S | 1.578 | 1.178 | 0.959 | 3.495 | 3.778 | 3.510 |
| MnO₂@F•S | 1.038 | 0.931 | 0.901 | 4.246 | 8.404 | 4.216 |
| PC | 0.936 | 0.867 | 0.891 | 8.360 | 14.981 | 11.425 |

The activation of STING and TLR4 shows that the modified inactivated bacteria in the present application effectively activate the natural immune system, help relieve the immune suppression of the tumor microenvironment, thereby strengthening the body's anti-tumor immune response. Based on Example D1 and Example D2, the modified bacteria in the present application have the effect of multi-pathway agonists while strengthening non-specific immune responses and anti-tumor immune responses, which helps to enhance anti-tumor immunotherapy.

### Example E

### Experiments on Treatment of Colon Cancer Tumor Model Using Different Metal Compounds Modified Bacteria

Group:
Group 1: Blank: blank control group;
Group 2: F.SA: inactivated Staphylococcus aureus
Group 3: F.E: inactivated Escherichia coli;
Group 4: F.L: inactivated lactobacilli;
Group 5: MnO₂@F.SA: inactivated Staphylococcus aureus modified with manganese dioxide
Group 6: MnO₂@F.E: inactivated Escherichia coli modified with manganese dioxide
Group 7: MnO₂@F.L: inactivated lactobacilli modified with manganese dioxide
Group 8: MnO₂@F.S: inactivated Salmonella modified with manganese dioxide

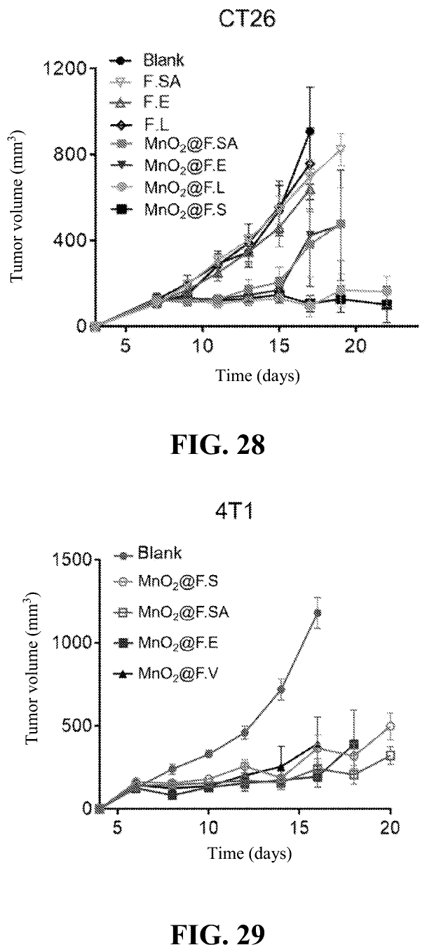

Different types of bacteria were prepared into modified bacteria according to the preparation method of Example A5, and a mouse subcutaneous CT26 colon cancer model treatment experiment was performed. The tumor inhibition rate of each group on the 17th day after seeding was calculated. The tumor inhibition rate of the blank control group was 0. The results of the tumor inhibition rate of other groups are shown in Table 3. The tumor growth curves are shown in FIG. 28. The results show that different types of modified inactivated bacteria can effectively inhibit tumors, and the tumor inhibition rate is about 5 times that of bacteria alone. It shows that the different types of modified bacteria in the present application can all achieve the treatment of tumors, and the type of bacteria has no direct impact on the efficacy of the modified bacteria.

TABLE 3
The statistics of tumor inhibition rates of the groups on the 17th
day after tumor inoculation in the experiment on mice treated with
bacteria modified or not modified with manganese compounds

| Group | F.SA | F.E | F.L | F.S |
|---|---|---|---|---|
| Tumor | 14.58 | 12.55 | 17.22 | 10.23 |
| inhibition | | | | |
| rate (%) | | | | |
| Group | MnO₂@F.SA | MnO₂@F.E | MnO₂@F.L | MnO₂@F.S |
| Tumor | 67.08 | 62.55 | >100 | >100 |
| inhibition | | | | |
| rate (%) | | | | |

### Example F

### Anti-Tumor Immune Memory Effect of the Body After Treatment of Tumors with Modified Bacteria

To verify the vaccine effect of the modified bacteria in the present application, a mouse colon cancer tumor model was established and treated with manganese dioxide-modified attenuated Salmonella (MnO₂@F.S). Finally, the subcutaneous tumors of the mice completely disappeared. On the 60th day after treatment, flow cytometry was used to analyze the proportion of memory T cells in the peripheral blood of mice. In mice whose tumors were cured by modified bacteria, the average proportion of memory T cells relative to all T cells in peripheral blood leukocytes was 83.86%, significantly increased compared with the blank control group without any treatment (the average content of memory T cells accounted for 59.5% of all T cells in peripheral blood leukocytes in mice), indicating that the modified bacteria in the present application can induce the generation of memory T cells to produce immune memory effects.

The cured mice were inoculated with the same type of tumor cells again, and the survival of the mice with the tumor was observed. The mouse survival curve is shown in FIG. 7. The mice cured by the modified bacteria did not develop symptoms after being inoculated with tumor cells again, and showed no cancer recurrence, can still have a long survival. In other words, the modified bacteria can inhibit the recurrence of tumors and produce a vaccine-like effect.

### Example G

### Study on Free-Drying Protective Agents for Modified Bacteria

The modified bacterial suspensions obtained in Examples A1-A3 and Example A5 were mixed with various additives (such as freeze-drying protective agents, excipients) and then freeze-dried, and we observed the state after freeze-drying and whether it can be re-dispersed into a bacterial suspension after adding the solvent (water). The observations on manganese sulfide-modified Salmonella mixed with the freeze-drying protective agent in different ratios and then freeze-dried are recorded in Table 4. For re-dispersible samples, their freeze-drying protective agents and corresponding proportions are defined as the available range. For samples with good freeze-dried morphology and capable of being re-dispersed into suspensions and samples with more regular morphology after freeze-drying that are helpful to actual production, packaging and ease of use, their freeze-drying protective agents used and corresponding proportions are set as the preferred range.

Among others, sucrose and β-cyclodextrin can make the modified bacteria have better freeze-drying effect. In addition to sucrose and β-cyclodextrin, trehalose, inositol, raffinose, inulin, dextran, maltodextrin, maltopolysaccharide can all enable the samples to re-suspend after freeze-drying without changing their properties, whereas lactose and mannitol cannot achieve effective protection against freeze-drying. All additives used in the examples were purchased commercially.

TABLE 4
Records of freeze-drying protection conditions
and status after freeze-drying of MnS@F.S

| Freeze-drying | Mass/volume | Morphology after | Whether it can be |
|---|---|---|---|
| protective agent | fraction (%) | freeze-drying | redistributed |
| Sucrose | 0.1 | Amorphous | Yes |
| | 0.5 | Amorphous | Yes |
| | 1 | Regular pie shape | Yes |
| | 5 | Regular pie shape | Yes |
| | 10 | Irregular block | Yes |
| | 20 | Irregular block | Yes |
| β-cyclodextrin | 0.1 | Regular pie shape | Yes |
| | 0.5 | Regular pie shape | Yes |
| | 1 | Regular pie shape | Yes |
| | 5 | Regular pie shape | Yes |
| | 10 | Irregular block | Yes |
| | 20 | Irregular block | Yes |
| Lactose | 0.1 | Regular pie shape | Yes |
| | 0.5 | Regular pie shape | Yes |
| | 1 | Regular pie shape | Yes |
| | 5 | Regular pie shape | Yes |
| | 10 | Irregular block | No |
| | 20 | Irregular block | No |

### Example H

### Example H1

### Synthesis of Manganese Bacteria with Different Feed Rations

According to the synthesis method of Example A5, in step S4, the loading amount of inactivated bacteria was fixed, manganese chloride of different qualities was added to the inactivated bacterial suspension, and the stability of the synthesized sample was observed. Since the entire reaction occurs in a liquid environment, and the product is dispersed in the liquid phase, so the stability of the sample is mainly indicated by whether the product has good dispersibility. If there is precipitation or agglomeration visible to the naked eye, it is considered that the product has poor dispersibility. On the contrary, if the liquid color of the product is uniformly distributed and no precipitation occurs, the product is considered to have good dispersibility. The results are shown in Table 5. From the perspective of product stability, when the loading amount of bacteria is about 1 billion bacteria and the loading amount of manganese chloride is no more than 13.5 mmol, manganese-biomineralized inactivated bacteria with good dispersibility can be obtained.

TABLE 5
Product stability corresponding to different loading amounts
of bacteria and manganese chloride after adding NaOH

| F.V loading amount | | MnCl₂ loading amount | |
|---|---|---|---|
| (number of cells) | | (μmol) | Product dispersibility |
| 0 | | 0.2 | Precipitated |
| | | 10 | |
| | | 20 | |
| 1 | billion | 0 | Good |
| | | 0.3 | Good |
| | | 0.6 | Good |
| | | 1.2 | Good |
| | | 2.4 | Good |
| | | 5 | Good |
| | | 7.5 | Good |
| 10 | million | 0.125 | Good |
| | | 1.875 | Good |
| | | 2.5 | Good |
| | | 6.25 | Good |
| | | 13.5 | Good |
| | | 13.75 | Precipitated, not well |
| | | | dispersed |

### Example H2

### Selection of Freeze-Drying Protective Agent

The manganese-biomineralized bacteria product of Example A5 was taken, different concentrations of freeze-drying protective agents were added according to the characteristics of different freeze-drying protective agents, the re-dissolution of the manganese-biomineralized bacteria after freeze-drying was observed, and appropriate types and concentrations of freeze-drying protective agents was screened. The results are shown in Table 6. When no freeze-drying protective agent was added, the freeze-dried manganese-biomineralized bacteria could not be re-dispersed in the solvent (water). After adding an appropriate proportion of a freeze-drying protective agent, a good freeze-drying re-dissolution effect could be achieved. The “mass/volume fraction” mentioned in Table 6 refers to the mass/volume fraction of the freeze-drying protective agent in the solution of the liquid preparation before freeze-drying.

TABLE 6
Re-dissolution of manganese-biomineralized bacteria MnO₂@F.S
after freeze-drying under different freeze-drying protective agents

| Freeze-drying | Mass/volume | Dispersibility after freeze-drying |
|---|---|---|
| protective agent | fraction (%) | and re-dissolution |
| Trehalose | 0 | Cannot be re-dispersed |
| | 1 | Good |
| | 5 | |
| | 10 | |
| | 20 | |
| Sucrose | 1 | Good |
| | 5 | |
| | 7.5 | |
| | 10 | |
| | 20 | |
| Mannose | 0.1 | Cannot be re-dispersed |
| | 1 | Good |
| | 5 | |
| | 10 | |
| | 20 | |

### Example H3

### Preparation of Manganese-Biomineralize Living Bacteria

S1: An aqueous solution of manganese chloride with a concentration of 10 mM was prepared;
S2: Amplification and culture of bacteria, where 50 μL of a frozen bacterial suspension was inoculated into 6 mL of fresh meat extract peptone (LB) medium, the cell culture tube was obliquely put into a constant-temperature shaker at 37° C. for activation at a shaking frequency of 220r/min for 12 h; then the activated bacterial suspension was diluted and coated on an LB solid medium plate, placed in a digital incubator to culture at 37° ° C.for 12 h. Single colonies in good condition were picked and inoculated into 5 mL of fresh LB broth medium, and placed on a constant temperature shaker at 37° C. to culture overnight to obtain a bacterial suspension;
S3: 50 μL of the bacterial suspension obtained in S2 was added to 20 mL of fresh LB liquid medium, the cell culture tube was obliquely put into a constant-temperature shaker at 37° C. for activation at a shaking frequency of 220 r/min for 12 h; the bacterial cells were collected by centrifugation, re-suspended in sterile normal saline, and centrifuged again; the bacterial cells were washed twice with sterile normal saline, and finally, the bacteria were re-suspended in 5-8 mL of normal saline so that the bacterial suspension was 24MCF, the absorbance of the bacterial suspension at a wavelength of 600 nm was 3, to obtain a bacterial suspension;
S4: A 10 mM manganese chloride solution and an appropriate amount of normal saline were added to the bacterial suspension and stirred for 15 min, and then a NaOH solution was added to adjust the pH of the solution to 10.0-11.0; the mixture was further stirred for 1 h, centrifuged to collect the precipitate, which was washed twice with sterile normal saline, and finally re-suspended in normal saline and stored at 4° C.

### Example H4

### Preparation of Manganese-Biomineralized Living Bacteria

The preparation method is the same as that of Example A5, the only difference is that when the pH value was adjusted to 8-10, the manganization time was increased by 30% to 100% compared to Example A5, but in the resultant manganese-biomineralized bacterial sample, the manganese compound particles on the surface of bacteria were finer.

### Example H5

### Preparation of Manganese-Biomineralized Living Bacteria

The preparation method is the same as Example A5, the only difference is that when the pH value was adjusted to 11-13, as the pH increased, the manganization time shortened, but when the pH was higher than 13, the bacterial morphology would be destroyed, which is not conducive to the uniformity of the final product.

### Example I

### Example I1

### Effect of Manganese-Biomineralized Bacteria on Cell Activity

CT26 tumor cells were inoculated in a 96-well plate at a density of 10⁴ cells/well and cultured in a cell incubator at 37° C. overnight. When the cells became adherent and were in good condition, different samples with different concentrations were added for co-incubation. The samples added in the groups and their concentrations are shown in Table 7.

After incubation for 24 h, the medium containing the sample was removed, the excess sample was washed away with PBS, and the relative activity of the cells was detected by MTT assay. The results are shown in Table 7. At a concentration of 2 μg/mL manganese dioxide and/or 0.12 MCF (McFarland units, indicating bacterial turbidity, 1 McFarland unit is about 300 million bacteria) (about 36 million) inactivated Salmonella bacteria as used in the cell experiments, no obvious toxicity was seen in all treatment groups, the cell viability was close to 100%, which shows the good safety of the samples in the present application. After continuing to increase the concentration to 10 times the dose of the cell experiment (20 μg/mL manganese dioxide), the MnO₂ treatment group and the MnO₂@F.S group began to show minor cytotoxicity, while the F.S group still had no toxicity. In addition, the experimental results show that compared with MnO₂@F.S, the MnO₂ & F.S treatment group showed great toxicity at a concentration of 5 μg/mL, with only 65.3% cell viability, which further proves that manganese dioxide grown on the surface of bacteria can effectively reduce the cytotoxicity caused by manganese dioxide, and the overall safety of manganese-biomineralized bacteria (MnO₂@F.S) is better than that of the mixture of bacteria and manganese dioxide at the same dose.

TABLE 7
Detection of relative cell activity

| | | | Relative | | | | Relative |
|---|---|---|---|---|---|---|---|
| | MnO₂ | | cell activity | | MnO₂ | | cell activity |
| Sample | concentration | | (%) | Sample | concentration | | (%) |
| MnO₂ | 2 | μg/mL | 103.2298 | MnO₂@F.S | 2 | μg/mL | 100.9358 |
| | 5 | μg/mL | 101.1529 | | 5 | μg/mL | 99.53656 |
| | 10 | μg/mL | 96.54142 | | 10 | μg/mL | 94.74153 |
| | 20 | μg/mL | 89.94985 | | 20 | μg/mL | 87.20143 |
| | 40 | μg/mL | 58.26804 | | 40 | μg/mL | 55.36007 |
| | 80 | μg/mL | 49.81624 | | 80 | μg/mL | 44.81117 |
| F.S | 36 | million | 95.51337 | MnO₂ & F.S | 2 | μg/mL | 96.13965 |
| (number | 90 | million | 103.2175 | | 5 | μg/mL | 65.34327 |
| of cells) | 1.9 | million | 105.205 | | 10 | μg/mL | 52.16201 |
| | 3.6 | million | 100.9091 | | 20 | μg/mL | 43.68012 |
| | 720 | million | 97.12801 | | 40 | μg/mL | 47.37861 |
| | 1.44 | billion | 91.28393 | | 80 | μg/mL | 38.59958 |

### Example I2

### Manganese-Biomineralized Inactivated Bacteria Promote Phagocytosis of DC2.4

Bacteria were labeled with Cy5.5 to obtain inactivated bacteria and manganese-biomineralized inactivated bacteria with fluorescent signals. DC2.4 cells in good condition were co-incubated with inactivated bacteria or manganese-biomineralized inactivated bacteria with fluorescent signals for a period of time, wherein the sample was quantified by the concentration of manganese ions, and the final concentration of the sample was 2 μg/mL. DC2.4 cells will phagocytose inactivated bacteria and manganese-biomineralized inactivated bacteria. At different time points, the fluorescence signal intensity of DC2.4 cells was detected by flow cytometry. The results are shown in FIG. 9. The abscissa of the spectrum is the fluorescence intensity, the peak area is the cell distribution density, and the statistical graph shows the statistics of the average fluorescence intensity. Compared with blank cells, after co-incubation of DC2.4 cells with fluorescently labeled manganese-biomineralized bacteria, fluorescence signals could be detected by flow cytometry. According to the statistical diagram, it can be found that after co-incubation of manganese-biomineralized inactivated bacteria with DC2.4 cells, the amount of manganese-biomineralized inactivated bacteria phagocytosed by DC2.4 cells was significantly greater than that of non-manganese-biomineralized inactivated bacteria, indicating that more inactivated bacteria with manganese dioxide grown on the surface can be phagocytosed.

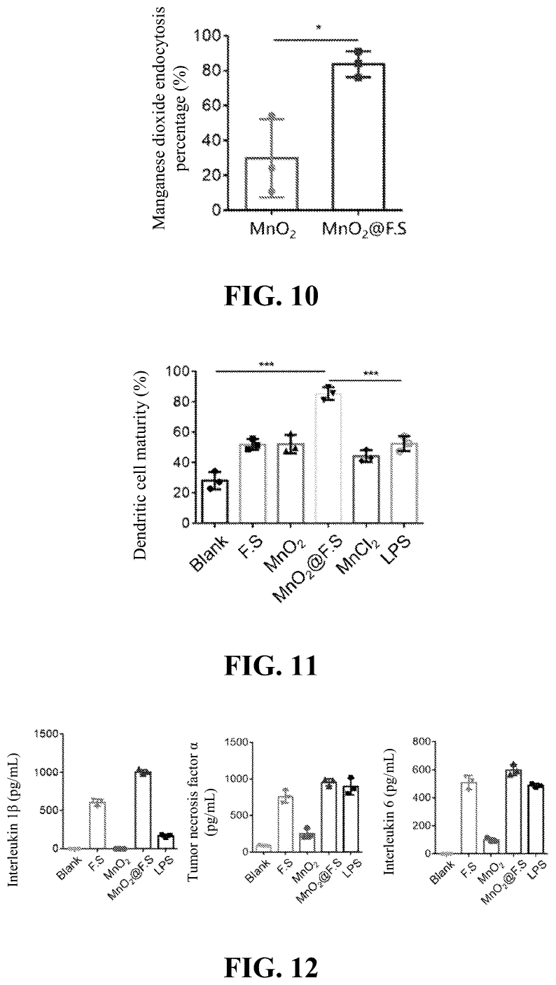

DC2.4 cells were co-incubated with manganese dioxide or manganese-biomineralized inactivated bacteria (with the same concentration of manganese element). After co-incubation for 12 h, ICP was used to detect the percentage of manganese ion content in the cells to the total input manganese ion content to observe the changes in the content of manganese internalized by cells. The statistics are shown in FIG. 10. The results show that manganese dioxide can be more phagocytosed by cells after growing on the surface of bacteria. During the same period of time, the manganese content in DC2.4 cells co-incubated with manganese-biomineralized inactivated bacteria was significantly higher than that in DC2.4 cells co-incubated with manganese dioxide. The above experimental results indicate that manganese-biomineralized inactivated bacteria can be phagocytosed by DC2.4 cells more effectively than manganese dioxide alone.

DC2.4 is a mouse bone marrow-derived dendritic cell line used to study the interaction between drugs and DC cells at the in vitro level. DC cells are the most powerful professional antigen-presenting cells in the body. Their function is to phagocytose, process and present antigens. Enhanced phagocytosis function helps DC cells take up more manganese dioxide, thereby stimulating the activation of the STING pathway more strongly. At the same time, the mature DC cells occurs during the process of phagocytosis/processing of antigens. More antigen uptake can promote the more maturation of dendritic cells, and mature DC cells can effectively activate naive T cells, triggering a series of anti-tumor immune responses.

### Example I3

### Stimulated Maturation of Bone Marrow-Derived DC Cells

Bone marrow-derived stem cells were extracted from the bone marrow of C57BL/6 mice, and colony-stimulating factor (GM-CSF) was added to promote the differentiation of stem cells into bone marrow-derived dendritic cells (BMDCs). BMDCs were co-incubated with different samples, which had a final concentration of 2 μg/mL as quantified by the manganese ions of manganese dioxide, and a final concentration of 0.12 MCF as quantified by F.S. After 12 h, the maturation percentages of BMDCs in different groups were detected. DC cells are efficient antigen-presenting cells in the body. They differentiate into mature DC cells after being stimulated by certain factors or ingesting antigens. Mature DC cells can effectively activate naive T cells, induce the generation of cytotoxic T lymphocytes, and secrete interleukin 1β (IL-1β), tumor necrosis factor α (TNF-α), interleukin 6 (IL-6), etc., so that they play a key role in the anti-tumor immune response. Tumor necrosis factor α (TNF-α) can kill or inhibit tumor cells; IL-1β is a pro-inflammatory cytokine. The secretion of IL-6 indicates that macrophages are polarized and can recruit more immune cells to tumor sites, causing a stronger anti-tumor immune response. IL-6 is mainly produced by macrophages and helper T cells, inducing cell differentiation and activating inflammatory responses. Therefore, samples that can effectively stimulate DC cell maturation can enhance the body's anti-tumor immune response.

The statistical results of the maturation percentage of DC cells detected are shown in FIG. 11. Correspondingly, the statistical results of the concentration of cytokines secreted by DC cells in different groups are shown in FIG. 12. Based on DC cell maturation percentage and cytokine secretion, it can be seen that the manganese-biomineralized inactivated bacteria can better stimulate the maturation of BMDCs, the results thereof in terms of various parameters were significantly different from the negative control group and even superior to the positive control group (2 μg/mL lipopolysaccharide, LPS), indicating that the manganese-biomineralized inactivated bacteria of the present application have excellent ability to stimulate DC cell differentiation and can effectively improve the efficiency of anti-tumor immune responses.

### Example I4

### Expression of Cytokines After Co-Incubation of Peritoneal Macrophages with Different Samples

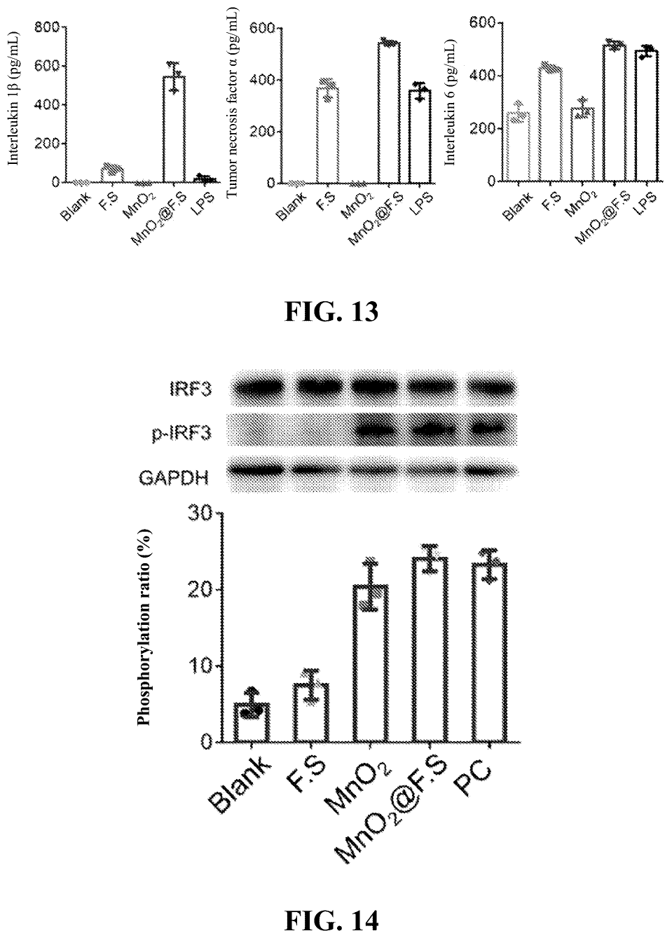

In addition to dendritic cells, macrophages also play an important role in anti-tumor immune responses. Macrophages extracted from the peritoneal cavity of C57BL/6 mice were co-incubated with different samples. The samples had a final concentration of 2 μg/mL as quantified by the manganese ions of manganese dioxide, and a final concentration of 0.12 MCF as quantified by F.S. The secretion of cytokines by macrophages was detected, and the results are shown in FIG. 13. The results of interleukin 1B, tumor necrosis factor α and interleukin 6 indicated that manganese-biomineralized inactivated bacteria have the strongest activation effect on macrophages, even better than the effect of the positive control. These three cytokines play an important role in anti-tumor immune responses. Among them, tumor necrosis factor a (TNF-α) can kill or inhibit tumor cells; IL-1β is a pro-inflammatory cytokine. The secretion of IL-6 indicates that macrophages are polarized and can recruit more immune cells to tumor sites, causing a stronger anti-tumor immune response. IL-6 is mainly produced by macrophages and helper T cells, inducing cell differentiation and activating inflammatory responses. This experiment shows that the activation of the innate immune response can also stimulate the proliferation and differentiation of cells involved in the immune response and increase the activity of immune cells (such as NK cells). These three cytokines can promote the anti-tumor immune response, indicating that the manganese-biomineralized inactivated bacteria of the present application contribute to the anti-tumor immune response and enhance the efficacy of tumor immunotherapy.

### Example I5

### Proving the Activation of STING Pathway by Manganese-Biomineralized Inactivated Bacteria at the Cellular Level

It is reported that manganese ions have the function of activating the STING pathway. In order to prove that manganese on the surface of manganese-biomineralized inactivated bacteria of the present application can also achieve similar effects as a STING stimulator, this example was designed. Human cervical cancer (HELA) cells were co-incubated with manganese-biomineralized inactivated bacteria, manganese dioxide, inactivated bacteria, and manganese chloride (positive control), respectively. The final concentration was 2 μg/mL as quantified by the manganese ions of manganese dioxide, and the final concentration was 0.12 MCF as quantified by F.S. After 24 h, western blot was used to detect the levels of interferon regulatory factor 3 (IRF3) and phosphorylated interferon regulatory factor 3 (p-IRF3), and the proportion of IRF3 phosphorylation was calculated. Interferon regulatory factor 3 (p-IRF3 represents phosphorylated IRF3). Phosphorylation of the 12C terminal of IRF-3 will induce the expression of IFNα/β. In this experiment, the degree of phosphorylation indicates the degree of activation of the STING pathway. GAPDH is a commonly used internal reference in western blot experiments and it is present in all cells. The internal reference band used in the experiment indicates that the amounts of cells added in this experiment are the same. The results are shown in FIG. 14. Compared with the control group, the proportion of phosphorylated interferon regulatory factor 3 in cells co-incubated with manganese-biomineralized inactivated bacteria was significantly increased, that is, the STING pathway activation of this group of cells was the highest; its STING activation level is close to that of the positive control (PC) 50 μM MnCl₂, indicating that manganese-biomineralized inactivated bacteria can effectively activate the STING pathway.

### Example I6

### Proving Activation of TLR4 Pathway by Manganese-Biomineralized Inactivated Bacteria Through Cell Experiments

Cells containing the Toll-like receptor 4 (TLR4) reporter gene (recorded as TLR4, TLR4-positive cells) were co-incubated with different samples (the experimental group was MnO₂@F.S, the blank control group was PBS, the general control group was F.S, the positive control group was PC, specifically 3 μg/mL MPLA), wherein the final concentration was 2 μg/mL as quantified by the manganese ions of manganese dioxide, and the final concentration was 0.12 MCF as quantified by F.S. The cells with TLR4 reporter gene will express luciferase after their TLR4 is activated, the luciferase can react with the luciferin substrate and emit fluorescence signals. After collecting the cell culture medium and adding the luciferin substrate, the fluorescence intensity was detected, and then the fluorescence intensity of each of the groups was compared with that of the PBS group. TLR4-negative cells (TLR4) do not express TLR4, so they will not be stimulated to express luciferase and will not produce fluorescence signals after luciferin substrate is added. This group of cells were used to prove that all the samples used in the experiment have no influence on the detection of fluorescein substrate and fluorescence signals.

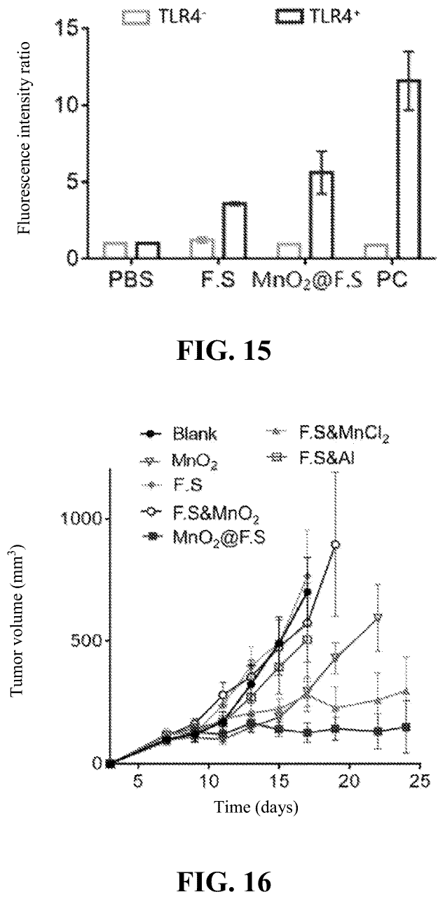

The fluorescence intensity statistics in different groups of cell culture media are shown in FIG. 15. Among them, manganese-biomineralized inactivated bacteria can significantly stimulate TLR4, and its ability to stimulate TLR4 is better than inactivated attenuated Salmonella. It shows that the manganese-biomineralized inactivated bacteria of the present application have an effect similar to TLR4 agonists.

The activation of STING pathway can obviously enhance the anti-tumor immunity of the body, and TLR4 is one of the receptors for the natural immune system to recognize pathogenic microorganisms. The activation of STING and TLR4 shows that the manganese-biomineralized inactivated bacteria in the present application effectively activate the natural immune system, help relieve the immune suppression of the tumor microenvironment, thereby strengthening the body's anti-tumor immune response.

Based on Example 15 and Example 16, the manganese-biomineralized inactivated bacteria in the present application have the effect of multi-pathway agonists, while strengthening non-specific immune responses and anti-tumor immune responses, which helps to enhance anti-tumor immunotherapy.

### Example J

### Animal Experiment

### Example J1

### Efficacy of Manganese-Biomineralized Inactivated Bacteria On Tumors as Compared with Inactivated Bacteria Combined with Different Immune Adjuvants

Grouping and Treatment Methods:
(1) Blank control group (Blank): intratumoral injection of normal saline;
(2) Manganese dioxide (MnO₂): intratumoral injection of manganese dioxide;
(3) Inactivated Salmonella (F.S): intratumoral injection of inactivated Salmonella;
(4) Mixture of inactivated Salmonella and manganese dioxide (F.S & MnO₂): intratumoral injection of a mixture of inactivated Salmonella and manganese dioxide;
(5) Inactivated Salmonella with manganese dioxide growing on the surface (MnO₂@F.S): intratumoral injection of manganese-biomineralized inactivated bacteria;
(6) Mixture of inactivated Salmonella and manganese chloride (F.S & MnC12): intratumoral injection of a mixture of inactivated Salmonella and manganese chloride;
(7) Mixture of inactivated Salmonella and aluminum adjuvant (F.S & Al): intratumoral injection of a mixture of inactivated Salmonella and an aluminum adjuvant;

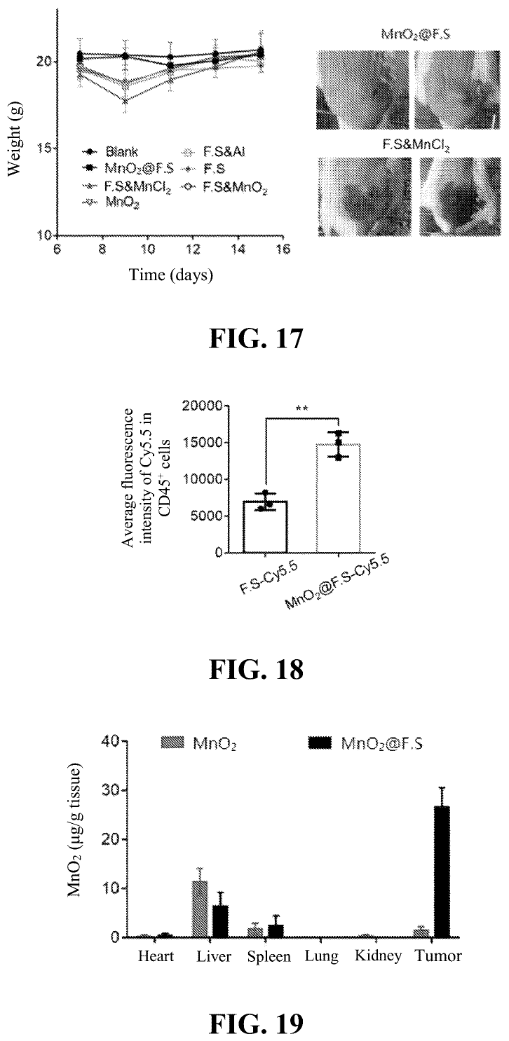

The CT26 colon cancer subcutaneous tumor model was inoculated on the backs of mice. When the tumor volume reached about 100 mm³, the mice were randomly divided into groups, and different samples were injected into the tumors in the mice according to the grouping. The doses of manganese and bacteria were controlled to be consistent among different groups, and the dose of manganese was 1 mg/kg. The tumor volume size and weight changes of mice were recorded every two days, and tumor growth curves and weight change curves were plotted. The results are shown in FIG. 16 and FIG. 17. FIG. 16 shows the tumor volume change curves. Injection of manganese-biomineralized inactivated bacteria had the best inhibitory effect on tumor growth in mice, injection of a mixture of inactivated bacteria and manganese dioxide or one of the components could not achieve this effect, and the mixture had no effect on tumor growth inhibition, indicating that only the manganese-biomineralized bacteria obtained after manganese dioxide grows on the surface of inactivated bacteria (MnO₂@F.S) can exert the most ideal effect, that is, the modified bacteria of the present application have good anti-tumor therapeutic effects.

The aluminum adjuvant was purchased commercially with Article No. 77161 and brand Thermo Scientific.

Mixing inactivated bacteria with manganese chloride and then injecting the mixture into the tumor can inhibit tumor growth to a certain extent, but its “therapeutic effect” is mainly attributable to the direct toxicity of manganese chloride. The results of weight change of mice and the photos of mice after injection of samples, manganese chloride caused large-scale necrosis of tumors and even surrounding tissues, and moreover, the weight of mice injected with manganese chloride dropped significantly, indicating that the strategy of mixing inactivated bacteria and manganese chloride to treat tumors is not ideal.

Aluminum adjuvant is a widely recognized immune adjuvant. It is reported that aluminum adjuvant can effectively enhance immune response. As one of the control groups, inactivated bacteria and aluminum adjuvant were mixed and injected into the tumor, which was not able to inhibit tumor growth.

Taken together, intratumoral injection of manganese-biomineralized inactivated bacteria with manganese dioxide growing on the surface can effectively inhibit tumor growth while ensuring safety.

### Example J2

### Phagocytosis of Samples by CD45 Cells at Tumor Sites

Bacteria were labeled with Cy5.5 to obtain inactivated bacteria and manganese-biomineralized inactivated bacteria with fluorescent signals. Fluorescently labeled manganese-biomineralized inactivated bacteria and inactivated bacteria were injected into the tumor, and 24 h later, the tumor tissue was taken and processed to obtain a cell suspension. The CD45⁺ cells among the cells at the tumor site were labeled with flow cytometry antibodies, and the fluorescence signal intensity of phagocytosed bacteria in CD45⁺ cells was detected and statistically analyzed, and the results are shown in FIG. 18. The abscissa of the spectrum is the fluorescence intensity, the peak area is the cell distribution density, and the statistical graph shows the statistics of the average fluorescence intensity. Compared with the control group, in the mice injected with manganese-biomineralized inactivated bacteria, the CD45⁺ cells in the tumor tissue cell suspension obtained by lysis had stronger fluorescence signals. According to the statistical diagram, it can be found that the amount of manganese-biomineralized inactivated bacteria phagocytosed by CD45⁺ cells at the tumor site was significantly larger than that of non-manganese-biomineralized inactivated bacteria, indicating that inactivated bacteria with manganese dioxide growing on the surface can be phagocytosed more by leukocytes in vivo. CD45⁺ is a cell marker of leukocytes. CD45⁺ cells are leukocytes. The enhanced phagocytosis of manganese bacteria by leukocytes helps to recruit more immune cells to the tumor site and indiscriminately phagocytose more tumor-related antigens, which is beneficial to the activation of the immune response at the tumor site and enhancing anti-tumor specific immune responses.

### Example J3

### Systemic Distribution of Sample After Injection

Manganese-biomineralized inactivated bacteria and manganese dioxide of the same dose were injected into the tumors of CT26 tumor-bearing mice. 24 h later, the heart, liver, spleen, lung, kidney and tumor tissues were taken, weighed and ground, and the manganese content in the tumor tissues and the organs was quantified using the ICP method. The statistical experimental results are shown in FIG. 19. Compared with injecting manganese dioxide alone, manganese dioxide biomineralized on bacteria can remain at the tumor site more, which will continue to provide immune stimulation to immune cells in the tumor microenvironment. In the liver, an important organ for manganese metabolism, it was observed that the treatment group with manganese dioxide biomineralized on bacteria had lower manganese content, indicating that the manganese-biomineralized bacterial samples would not burden the liver in a short period of time, further validating the advantage in safety of the manganese-biomineralized bacterial sample.

### Example J4

### Changes in Oxygen Content in Tumor Sites After Sample Injection

To prove that manganese-biomineralized inactivated bacteria can effectively improve the hypoxic state of the tumor microenvironment, changes in oxygen content in the tumor microenvironment in tumor-bearing mice were detected using oxygen content detection probes 1 h and 4 h after sample injection, respectively.

Grouping and Treatment Methods:
(1) Blank control group (Blank): intratumoral injection of normal saline;
(2) Manganese dioxide (MnO₂): intratumoral injection of manganese dioxide;
(3) Inactivated Salmonella (F.S): intratumoral injection of inactivated Salmonella;

(4) Manganese-biomineralized inactivated Salmonella (MnO₂@F.S): intratumoral injection of manganese-biomineralized inactivated Salmonella.

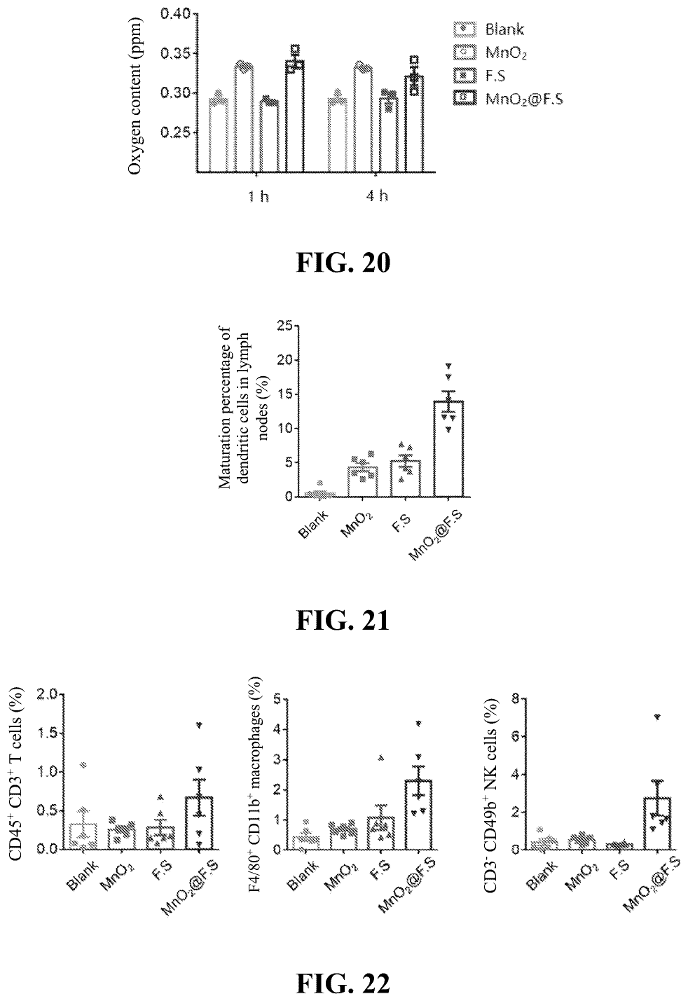

The statistical data of the experimental results are shown in FIG. 20. The oxygen content in the untreated tumor tissue was about 0.28 ppm, indicating a hypoxic state. 1 h after the sample was injected into the tumor, the oxygen content in both the manganese dioxide treatment group and the manganese-biomineralized inactivated bacteria treatment group increased to about 0.34 ppm, the oxygen content at the tumor site was significantly increased. A similar trend could still be observed after 4 h. This shows that manganese-biomineralized bacteria can effectively improve the hypoxic state of the tumor microenvironment through the action of manganese dioxide. The hypoxic tumor microenvironment is a typical immunosuppressive environment. The improvement of the hypoxic microenvironment is conducive to the subsequent infiltration of immune cells into the lesion and their activation in this environment to exert immunotherapeutic effects.

### Example J5

### Manganese-Biomineralized Inactivated Bacteria Strategy can Effectively Activate the Anti-Tumor Immune System

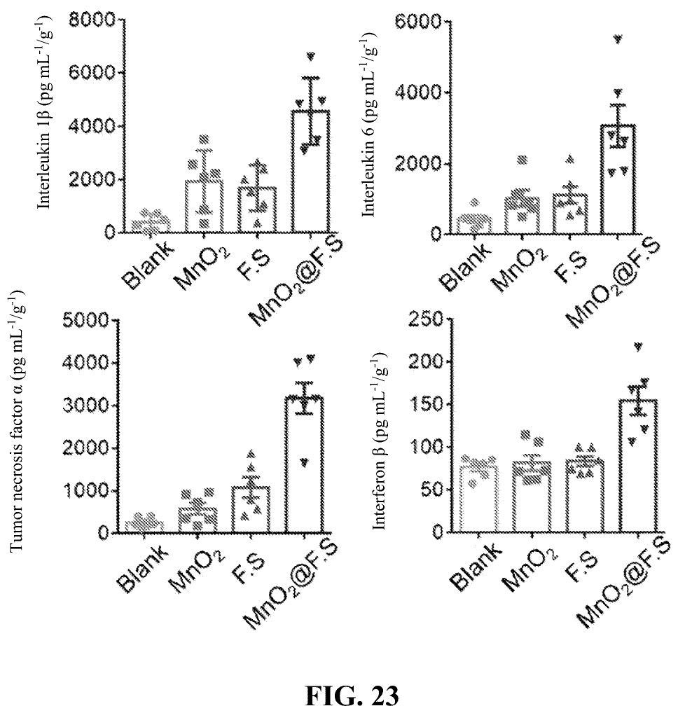

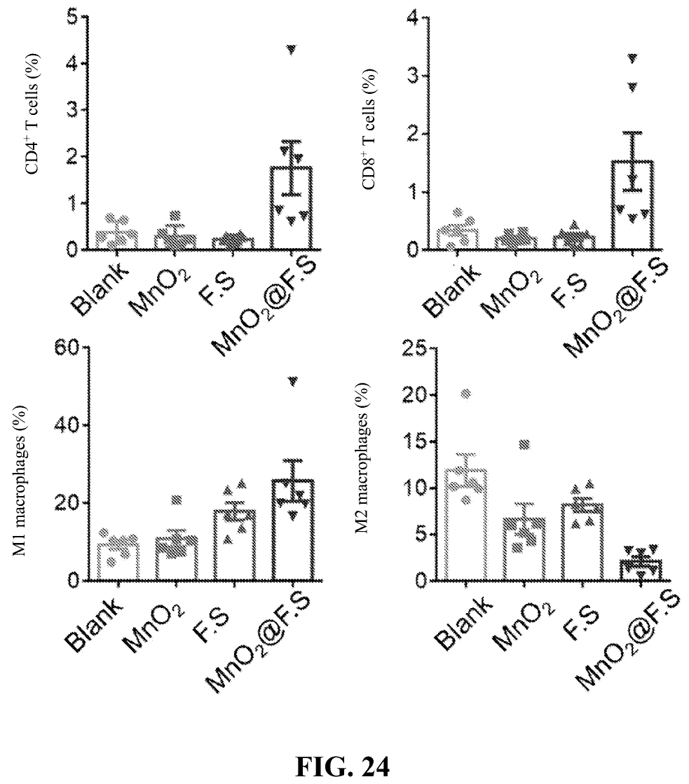

This example aimed to prove that manganese-biomineralized bacteria can effectively activate the innate immune system and adaptive immune system, improve the tumor immunosuppressive microenvironment, and effectively enhance the anti-tumor immune response. 24 h after intratumoral injection of different samples in CT26 tumor-bearing mice, mouse lymph nodes were taken to detect the maturity of dendritic cells (FIG. 21), and tumor tissues were taken to detect the contents of cytokines therein (FIG. 23). After treatment for 24 h and 5 days, the tumor tissue was taken to detect immune cell infiltration (FIG. 22 and FIG. 24).

Grouping and Treatment Methods:
Group 1: Blank, blank control group (intratumoral injection of normal saline);
Group 2: MnO₂, manganese dioxide (intratumoral injection of manganese dioxide);
Group 3: F.S, inactivated Salmonella (intratumoral injection of inactivated Salmonella);
Group 4: MnO₂@F.S, manganese-biomineralized inactivated Salmonella (intratumoral injection of manganese-biomineralized inactivated Salmonella).

The maturation of lymph node dendritic cells is an important step in immune activation. Immature dendritic cells migrate to lymph nodes after ingesting antigens and gradually become mature during migration to express costimulatory molecules and present antigens to T cells in lymph nodes. The lymph nodes on the tumor side were removed 24 h after sample injection, and then the proportion of mature dendritic cells among all dendritic cells was detected using a flow cytometer. The statistical results are shown in FIG. 21. Among the four groups, the mice in the group treated with manganese-biomineralized inactivated bacteria had the highest DC maturity level, which was 26.9 times that in the group injected with normal saline. This indicates that manganese-biomineralized inactivated bacteria have excellent immune stimulating ability and can effectively activate the immune system of mice.

The tumor microenvironment is an immunosuppressive microenvironment where the immune response is not easily activated, resulting in extremely low contents of cytokines. To prove the activation of the immune response and the improvement of immune suppression by the samples of manganese-biomineralized bacteria, mouse tumor tissues were removed at 24 h, ground, and detected for the contents of interleukin 1B, interleukin 6, tumor necrosis factor a and interferon β therein by enzyme-linked immunoassay. The results are shown in FIG. 23. In mice injected intratumorally with manganese-biomineralized inactivated bacteria, the levels of interleukin 1β, interleukin 6, tumor necrosis factor a, and interferon β at the tumor site were significantly higher than those in other control groups, showing the superiority of the strategy of manganese-biomineralized inactivated bacteria in innate immune activation in the tumor microenvironment. Interferon β is a marker of activation of the STING pathway. Manganese dioxide in the manganese-biomineralized bacterial samples can be internalized by immune cells more (Example 12) and manganese-biomineralized inactivated bacteria can remain at the tumor sites longer than free manganese dioxide (Example J3), therefore these examples have the highest ability to activate the STING pathway.

The activation of the innate immune response (FIG. 22) and the activation of the specific immune response (FIG. 24) were evaluated and compared 24 h and 5 days after tumor treatment. Tumor tissues were removed at 24 h and 5 days, ground, and stained with flow cytometry antibodies. A flow cytometer was used to detect the infiltration of the innate immune system—macrophages and NK cells, as well as adaptive immune cells—T cells, as shown in FIG. 22. After 24 h, the percentages of macrophages and NK cells at the tumor site were significantly upregulated, indicating the activation of the innate immune system. The results are also consistent with FIG. 23. However, there was no significant difference in the T cells of the specific immune system among the four groups, which shows that macrophages and NK cells are mainly responsible for this stage. 5 days after treatment, the percentages of CD4⁺ T cells (helper T cells) and CD8⁺ T cells (killer T cells) in the tumors of mice in the manganese-biomineralized inactivated bacteria treatment group were significantly increased, proving that mice in this group entered the specific immune activation stage, while there were no significant differences in the other three groups of mice. (FIG. 24)

In addition, the macrophage polarization state also changed on day 5 in mice treated with manganese-biomineralized inactivated bacteria. M1 macrophages are of immune-activating type and can help with antigen presentation and T cell differentiation. M2 macrophages are of tumor-promoting type and are generally considered to be related to tumor metastasis. The experimental data showed that on the fifth day, the polarization state of macrophages at the tumor site of mice in the manganese-biomineralized inactivated bacteria group changed towards the direction of anti-tumor immunity. (FIG. 24)

In summary, after intratumoral injection of manganese-biomineralized inactivated bacteria, the tumor microenvironment is effectively improved, and cytokines related to immune activation are significantly upregulated, which helps immune cells such as macrophages and NK cells to infiltrate the tumor site, and also more dendritic cells absorb the antigen, migrate to the lymph nodes, and present the antigen to T cells in the lymph nodes, completing the transition from activation of the innate immune system to activation of the specific immune system. After T cells differentiate, they migrate to the tumor site. The percentages of helper T cells and killer T cells are significantly upregulated, specifically killing tumor cells and helping macrophages to polarize to the anti-tumor M1 type. The polarized macrophages in turn can continue to activate the anti-tumor function of T cells and complete the activation of specific immune responses.

### Example J6

### Experiment on Treatment of Tumor With Manganese-Biomineralized Bacteria After Blockade of NK cells

To prove that manganese-biomineralized inactivated bacteria can induce the activation of innate immunity, mouse natural killer cells (NK cells) were blocked, causing the loss of function of mouse NK cells. At the same time, IgG, as an antibody that does not affect NK cell function, was used as a control for NK antibodies in this example.

Group:
Group 1: blank: blank control group;
Group 2: MnO₂@F.S & IgG: treatment group of manganese-biomineralized inactivated bacteria combined with immunoglobulin;
Group 3: MnO₂@F.S & a-NK 1.1: treatment group of manganese-biomineralized inactivated bacteria combined with NK 1.1 cell antibody.

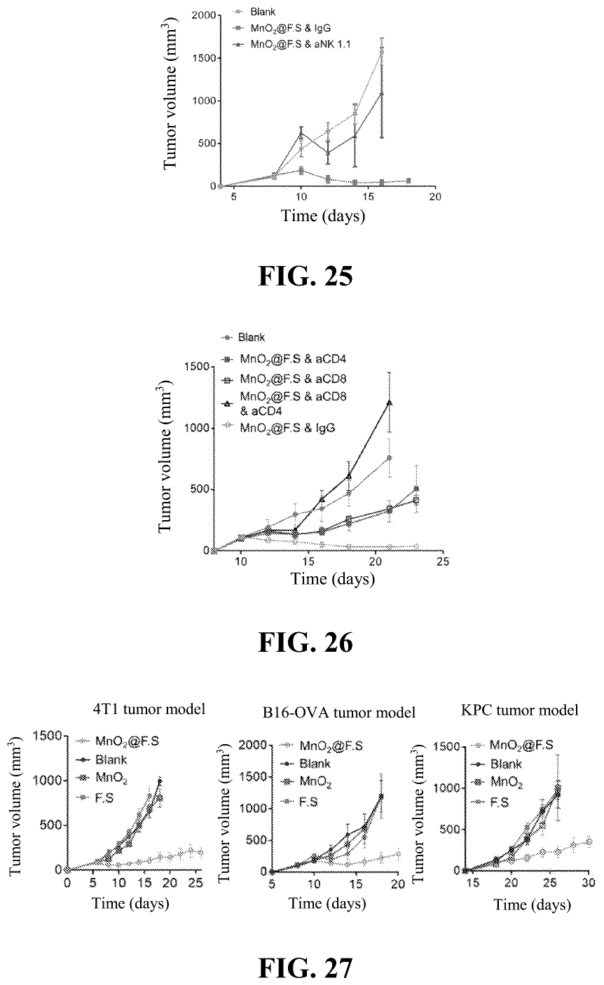

Melanoma B16-OVA tumor cells were inoculated on the backs of mice to construct a subcutaneous melanoma model. The treatment method involved intratumoral injection of manganese-biomineralized inactivated bacteria (MnO₂@F.S). The mice in the group 3 were injected with NK blocking antibody (a-NK 1.1) 1 day before starting tumor treatment and on days 2, 4, and 6 after dosing, and mice in the second group were injected with IgG 1 day before starting tumor treatment and on days 2, 4, and 6 after dosing. The tumor size was monitored every two days and tumor growth curves were plotted. The results are shown in FIG. 25. After the loss of function of NK cells, the manganese-biomineralized inactivated bacteria hardly inhibited tumor growth, indicating that this preparation can activate the innate immune response, and after activating the innate immune response, activated NK cells play an important role in inhibiting tumor growth.

### Example J7

### Experiment on Treatment of Tumor with Manganese-Biomineralized Bacteria After Blockade of T Cells

In order to prove that manganese-biomineralized inactivated bacteria can activate specific immune responses, CD4 antibodies and CD8 antibodies were used to block the action site on the surface of T cells, causing CD4⁺ CD8⁺ T cells to lose their function. At the same time, another group was injected with IgG as control.

Group:
Group 1: blank: blank control group;
Group 2: MnO₂@F.S & α-CD4: treatment group of manganese-biomineralized inactivated bacteria combined with anti-CD4 antibody;
Group 3: MnO₂@F.S & α-CD8: treatment group of manganese-biomineralized inactivated bacteria combined with anti-CD8 antibody;
Group 4: MnO₂@F.S & α-CD4 & α-CD8: treatment group of manganese-biomineralized inactivated bacteria combined with anti-CD4 and anti-CD8 antibodies;
Group 5: MnO₂@F.S & IgG: treatment group of manganese-biomineralized inactivated bacteria combined with immunoglobulin.

A mouse subcutaneous CT26 tumor model was established. When the tumor size was about 100 mm3, the mice were randomly divided into groups. Mice in different groups were injected intravenously with corresponding antibodies 1 day before intratumoral administration and on days 3, 5, and 7 after dosing. Tumor growth was recorded, and tumor growth curves were plotted. The results are shown in FIG. 26. As can be seen from the figure, in the same case of administering manganese-biomineralized inactivated bacteria , using antibodies against CD4 or CD8 to block the function of the corresponding CD4⁺ T cells or CD8⁺ T cells in mice can significantly reduce the tumor treatment effect, only when T cells function normally can a better therapeutic effect be achieved, indicating that the effectiveness of manganese-biomineralized inactivated bacteria depends on the activation of specific immune responses, and depends on the anti-tumor immune response mediated by T cells to inhibit tumor growth.

### Example J8

A mouse back 4T1 breast cancer tumor model, a B16-OVA melanoma model and a KPC pancreatic cancer model were respectively established, and then treated by intratumoral injection of different samples. The tumor growth curves were recorded. The results are shown in FIG. 27. The results show that all three tumor models can be well inhibited by manganese-biomineralized inactivated bacteria, but a single component cannot inhibit tumor growth. The results of this example confirm Example J1 and show that the manganese-biomineralized inactivated bacteria of the present application are effective against a variety of solid tumors.

### Example J9

### Experiments on Treatment of Breast Cancer Tumor Model Using Different Manganese-Biomineralized Bacteria

Group 1: blank: blank control group;
Group 2: MnO₂@F.S: manganese-biomineralized inactivated Salmonella;
Group 3: MnO₂@F.SA: manganese-biomineralized inactivated Staphylococcus aureus;
Group 4: MnO₂@F.E: manganese-biomineralized inactivated Escherichia coli;
Group 5: MnO₂@F.V: manganese-biomineralized inactivated Salmonella VNP20009.

A mouse subcutaneous 4T1 breast cancer model was established. Different types of manganese-biomineralized inactivated bacteria were injected into the tumor. The tumor growth was monitored and tumor growth curves were plotted. The results are shown in FIG. 29. It can be seen from the growth curves that all manganese-biomineralized bacteria can well inhibit tumor growth, which is consistent with the results of Example J3, indicating that the manganese-biomineralized inactivated bacteria of the present application are universally applicable to different tumors.

### Example J10

### The Therapeutic Effect of Manganese-Biomineralized Bacteria Prepared with Different Feed Ratios on Tumors

Group:

| | | Manganese ion | |
|---|---|---|---|
| | Bacteria loading | loading amount | Tumor inhibition |
| No. | amount | (μmol) | rate (%) |
| Group 1 | 1 billion | 0.125 | 74.59 |
| Group 2 | | 0.625 | 90.84 |
| Group 3 | | 2.5 | 78.86 |
| Group 4 | 0 | 0 | 0 |

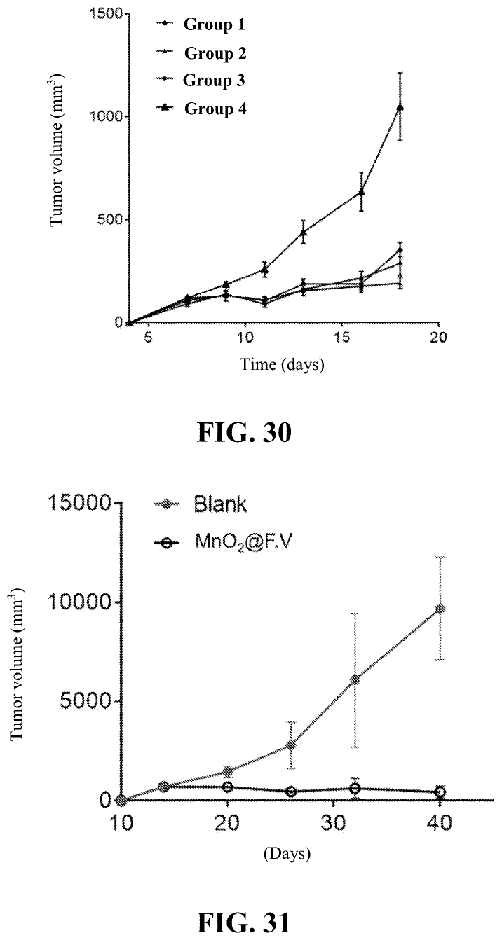

According to the method of Example J1, manganese-biomineralized bacteria with different feed proportions were prepared, a mouse CT26 subcutaneous tumor model was established, and mice were randomly divided into groups. The mice were intratumorally injected with corresponding drugs according to the group setting, the injection dose was 3.6*10⁸ manganese-biomineralized bacteria, and the corresponding manganese dioxide dosages were 3 μg, 16 μg, and 89 μg, respectively. Thereafter, the tumor size was measured once every two days and tumor growth curves were plotted. The results are shown in FIG. 30.

According to the analysis of the tumor growth curve, compared with the tumor growth curve of the blank group (group 4), manganese-biomineralized bacteria prepared with different feed ratios all had an inhibitory effect on tumor growth. This shows that the manganese-biomineralized bacteria prepared in the present application have a wide range of applicable feed ratios. In actual treatment, the optimal formula can be confirmed through more research.

### Example JI1

The New Zealand rabbit liver VX2 cancer model is one of the few liver cancer recurrence and metastasis models established in larger animals. It can better simulate the recurrence and metastasis of human liver cancer. Therefore, a New Zealand rabbit liver cancer model was established and the tumor was treated with the manganese-biomineralized inactivated bacteria MnO₂@F.V of the present application. The amount of bacteria used in sample preparation was 10.5 MCF, and the amount of manganese dioxide used was 160.7 μg. The results are shown in FIG. 31. Compared with the group without any treatment, the tumor lesions of New Zealand rabbits treated with manganese-biomineralized inactivated bacteria (MnO₂@F.V) were almost completely eliminated, indicating its excellent anti-tumor properties.

### Example J12

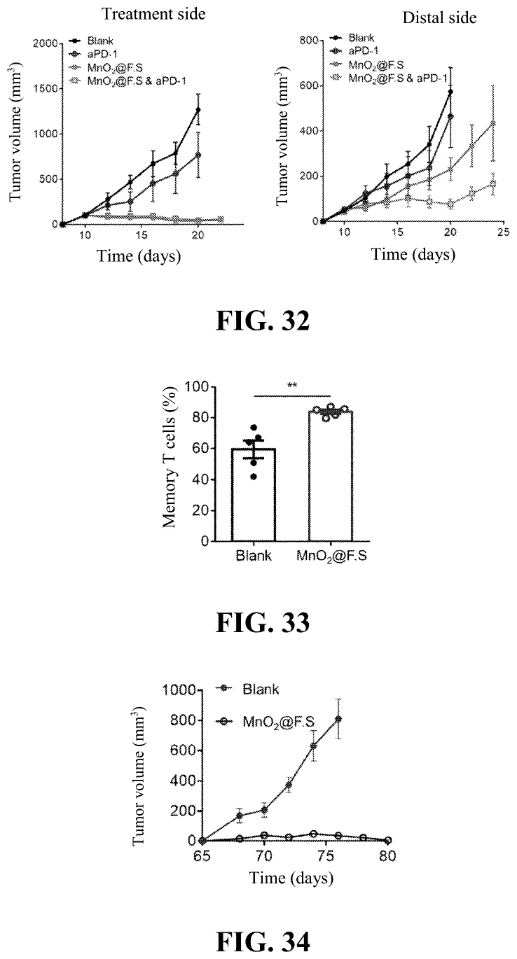

A mouse bilateral tumor model was established. The CT26 colon cancer subcutaneous tumor model was inoculated on the left and right sides of the mouse's back. The number of tumor cells inoculated on the left side was half that on the right side. The right tumor simulated the primary tumor, while the left tumor simulated metastases. When the volume of the right tumor reached about 100 mm³, the mice were randomly divided into groups. Different samples were injected into the tumor on the right side of the mice according to the corresponding groups, and the immune checkpoint anti-PD-1 (i.e., α-PD-1) antibody was injected into the tail vein of the two groups of mice on days 1, 3, and 5 after treatment. The volume of bilateral tumors in mice was recorded every two days, and tumor growth curves were plotted. The results are shown in FIG. 32.

Grouping and Treatment Methods:
Group 1: blank: injection of normal saline into the right tumor;
Group 2: α-PD-1: injection of immune checkpoint antibody anti-PD-1 into the tail vein for three times;
Group 3: MnO₂@F.S: injection of manganese-biomineralized bacteria into the right tumor;
Group 4: MnO₂@F.S & α-PD-1: injection of manganese-biomineralized bacteria into the right tumor and injection of immune checkpoint antibody anti-PD-1 into the tail vein for three times.

As shown in the results in FIG. 32, the tumor growth curve on the treatment side shows the excellent therapeutic effect of manganese-biomineralized bacteria. Compared with the group injected with immune checkpoint antibody (α-PD-1) alone, the therapeutic effect of manganese-biomineralized bacteria (MnO₂@F.S) was significantly better than that of immune checkpoint treatment. In the MnO₂@F.S group, the tumors on the treated side of the mice were almost completely suppressed. Observing the tumor growth of distal tumors, it was found that combined with immune checkpoints, the inhibitory effect of manganese-biomineralized bacteria on the growth of distal tumors could be further strengthened, and the growth rate of distal tumors slowed down. It shows that manganese-biomineralized bacteria can inhibit the growth of in situ tumors and induce a systemic anti-tumor immune response and has the effect of inhibiting the growth of distal tumors. Combined with immune checkpoint blockade therapy, manganese-biomineralized bacteria can more effectively inhibit the growth of distal tumors and have the potential of combination with checkpoint blockade therapy.

### Example J13

### Immune Memory Effect

In order to verify the vaccine effect of the manganese-biomineralized bacteria of the present application, a mouse CT26 colon cancer tumor model was established, and the unilateral tumor was treated according to the method described in Example J12. Finally, the subcutaneous tumors of the mice completely disappeared. On the 60th day after treatment, flow cytometry was used to analyze the proportion of memory T cells in the peripheral blood of mice. The statistical results are shown in FIG. 33. The content of memory T cells in mice with tumors cured by manganese-biomineralized bacteria increased significantly, indicating that the manganese-biomineralized bacteria of the present application can induce the generation of memory T cells to produce immune memory effect.

Moreover, the cured mice were inoculated with tumor cells again, and tumor growth was recorded. The results are shown in FIG. 34. The results show that mice cured by manganese-biomineralized bacteria had almost no tumor growth after being re-inoculated with tumor cells. In other words, manganese-biomineralized bacteria can cure tumors while also inhibiting tumor recurrence, producing a vaccine-like effect.

### Example K

### Example K1

### Preparation of Manganese-Biomineralized Inactivated Bacteria Adhered with Manganese Dioxide (Using Manganese Sulfate as Raw Material)

S1: An aqueous solution of manganese sulfate with a concentration of 10 mM was prepared;
S2: Amplification and culture of bacteria, where 50 μL of a frozen bacterial suspension of attenuated Salmonella was inoculated into 6 mL of fresh meat extract peptone (LB) medium, the cell culture tube was obliquely put into a constant-temperature shaker at 37° C. for activation at a shaking frequency of 220 r/min for 12 h; then the activated bacterial suspension was diluted and coated on an LB solid medium plate, placed in a digital incubator to culture at 37° C. for 12 h. Single colonies in good condition were picked and inoculated into 5 mL of fresh LB broth medium, and placed on a constant temperature shaker at 37° C. to culture overnight to obtain a bacterial suspension;
S3: The bacterial suspension obtained in S2 was added to 20 mL of fresh LB liquid medium, the cell culture tube was obliquely put into a constant-temperature shaker at 37° ° C.for activation at a shaking frequency of 220 r/min for 12 h; the bacterial cells were collected by centrifugation, re-suspended in sterile normal saline, and centrifuged again; then 5 mL of 4% paraformaldehyde was added to fix for 24 h, the fixed inactivated bacteria were collected by centrifugation, the bacterial cells were washed twice with sterile normal saline, and finally, the inactivated bacteria were re-suspended in normal saline so that the final bacterial suspension had OD₆₀₀=3, that is, the absorbance of the bacterial suspension at a wavelength of 600 nm was 3; an inactivated bacterial suspension was obtained, the inactivated bacterial suspension could be stored at 4° C.; 50 μL of the bacterial suspension was taken and spread on solid culture medium plate , and after culture, it was confirmed that the bacteria in the bacterial suspension had been completely inactivated;
S4: A 10 mM manganese sulfate solution and an appropriate amount of normal saline were added to the inactivated bacterial suspension and stirred for 15 min, and then sodium hydroxide was added to adjust the pH to 10-11; the mixture was further stirred for 1 h, centrifuged to collect the precipitate, which was washed twice with sterile normal saline, and finally re-suspended in 1 mL normal saline and stored at 4° C.

### Example K2

### Preparation of Manganese-Biomineralized Inactivated Bacteria Adhered with Manganese Sulfide

S1: An aqueous solution of manganese chloride with a concentration of 10 mM was prepared;
S2: Amplification and culture of bacteria, where 50 uL of a frozen bacterial suspension of attenuated Salmonella was inoculated into 6 mL of fresh meat extract peptone (LB) medium, the cell culture tube was obliquely put into a constant-temperature shaker at 37° C. for activation at a shaking frequency of 220 r/min for 12 h; then the activated bacterial suspension was diluted and coated on an LB solid medium plate, placed in a digital incubator to culture at 37° C. for 12 h. Single colonies in good condition were picked and inoculated into 5 mL of fresh LB broth medium, and placed on a constant temperature shaker at 37° C. to culture overnight to obtain a bacterial suspension;
S3: The bacterial suspension obtained in S2 was added to 20 mL of fresh LB liquid medium, the cell culture tube was obliquely put into a constant-temperature shaker at 37° C. for activation at a shaking frequency of 220 r/min for 12 h; the bacterial cells were collected by centrifugation, re-suspended in sterile normal saline, and centrifuged again; then 5 mL of 4% paraformaldehyde was added to fix for 24 h, the fixed inactivated bacteria were collected by centrifugation, the bacterial cells were washed twice with sterile normal saline, and finally, the inactivated bacteria were re-suspended in normal saline so that the final bacterial suspension had OD₆₀₀=3, that is, the absorbance of the bacterial suspension at a wavelength of 600 nm was 3; an inactivated bacterial suspension was obtained, which could be stored at 4° C.; 50 μL of the bacterial suspension was taken and spread on solid culture medium plate, and the absence of living bacteria in the bacterial suspension was confirmed;
S4: A 10 mM manganese chloride solution and an appropriate amount of normal saline were added to the inactivated bacterial suspension and stirred for 15 min, and then 0.1 M sodium sulfate was added; the mixture was further stirred for 1 h, centrifuged to collect the precipitate, which was washed twice with sterile normal saline, and finally re-suspended in 1 mL normal saline and stored at 4° C.

### Example K3

### Preparation of Manganese-Biomineralized Inactivated Bacteria by Reduction Method

SS1: A 10 mM aqueous potassium permanganate solution was prepared for later use;
SS2: Amplification and culture of bacteria, where 50 μL of a frozen bacterial suspension of attenuated Salmonella was inoculated into 6 mL of fresh meat extract peptone (LB) medium, the cell culture tube was obliquely put into a constant-temperature shaker at 37° C. for activation at a shaking frequency of 220r/min for 12 h; then the activated bacterial suspension was diluted and coated on an LB solid medium plate, placed in a digital incubator to culture at 37° C. for 12 h. Single colonies in good condition were picked and inoculated into 5 mL of fresh LB broth medium, and placed on a constant temperature shaker at 37° C. to culture overnight to obtain a bacterial suspension;
SS3: The bacterial suspension obtained in SS2 was added to 20 mL of fresh LB liquid medium, the cell culture tube was obliquely put into a constant-temperature shaker at 37° C. for activation at a shaking frequency of 220 r/min for 12 h; the bacterial cells were collected by centrifugation, re-suspended in sterile normal saline, and centrifuged again; then 5 mL of 4% paraformaldehyde was added to fix for 24 h, the fixed inactivated bacteria were collected by centrifugation, and the inactivated bacteria were re-suspended in normal saline so that the final bacterial suspension had OD₆₀₀=3, that is, the absorbance of the bacterial suspension at a wavelength of 600 nm was 3; an inactivated bacterial suspension was obtained, which could be stored at 4° C.; 50 μL of the bacterial suspension was taken and spread on solid culture medium plate, and the absence of living bacteria in the bacterial suspension was confirmed;
SS4: A 10 mM potassium permanganate solution and an appropriate amount of normal saline were added to the inactivated bacterial suspension and stirred for 1.5 h, centrifuged to collect the precipitate, which was washed twice with sterile normal saline, and finally re-suspended in 1 mL normal saline and stored at 4° C.

Depending on the volume of the potassium permanganate solution added, 0.2-3 μmol of manganese per 1 billion bacteria can be added to form a stable manganese-biomineralized bacterial suspension.

### Example K4

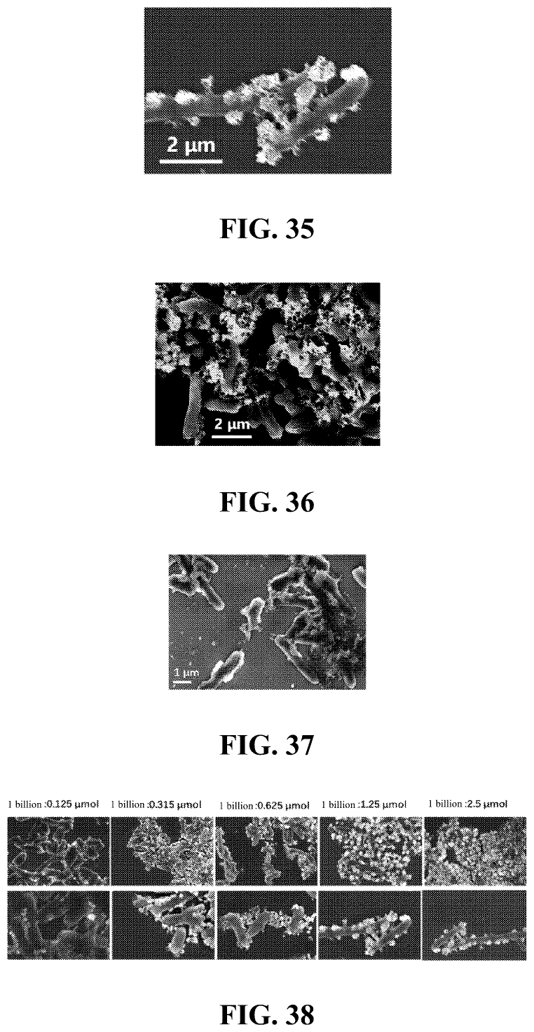

An appropriate amount of the final products of Examples K1-K3 were taken to prepare SEM samples, and the appearance and morphology of the samples were characterized by using a scanning electron microscope. The obtained electron microscopy images are shown in FIG. 35 (MnO₂@F.S prepared using manganese sulfate as raw material), FIG. 36 (manganese-biomineralized bacteria adhered with manganese sulfide (MnS@F.S) and FIG. 37 (MnO₂@F.S prepared by reduction method). The fixed inactivated bacteria maintained the appearance characteristics of the original bacteria, the surface of the manganese-biomineralized bacteria had a layer of rough precipitate, and the bacteria themselves maintained their original shape and size, indicating that the bacteria themselves are not destroyed when adhered with different metal compounds, but a layer of material is adhering to their surface. Various manganese bacteria can be obtained using different preparation methods.

### Example K5

The method of Example K1 was used to prepare bacteria with manganese oxide growing on the surface with different feed ratios. The raw materials were manganese sulfate and attenuated Salmonella. The morphology of the prepared finished product was observed with a scanning electron microscope. The results are shown in FIG. 38. The manganese-biomineralized bacteria obtained with different feed ratios had manganese oxide solids of relatively uniform size adhering to their surfaces. As the loading amount of manganese sulfate increased, the manganese oxide growing on the surface of bacteria increased.

The foregoing detailed description is provided by way of explanation and example, and is not intended to limit the scope of the appended claims.

Similarly, it should be noted that in order to simplify the presentation of the disclosure of the present application to facilitate understanding of one or more embodiments of the invention, in the foregoing description of the embodiments of the present application, multiple features are sometimes combined into one embodiment, accompanying drawing or descriptions thereof. However, this method of disclosure does not imply that the subject matter of the present application requires more features than are mentioned in the claims. In fact, embodiments may have less than all features of a single disclosed embodiment.

In some embodiments, numbers are used to describe the quantities of components and properties. It should be understood that such numbers used to describe the embodiments are modified by the modifiers “about”, “approximately” or “substantially” in some examples. Unless stated otherwise, “about,” “approximately,” or “substantially” indicates that the stated number is subject to ±variations. Accordingly, in some embodiments, the numerical parameters used in the specification and claims are approximations that may vary depending on the desired features of individual embodiments.

Finally, it should be understood that the embodiments described in the present application are only used to illustrate the principles of the embodiments of the present application. Other variations are possible within the scope of the present application. Accordingly, by way of example and not limitation, alternative configurations of the embodiments of the present application may be considered consistent with the teachings of the present application. Accordingly, embodiments of the present application are not limited to those expressly introduced and described herein.

What is claimed is:

1. (canceled)

2. A modified bacterium comprising a bacterium body and a poorly soluble or insoluble biologically acceptable metal compound modified on the surface of the bacterium body, wherein said metal compound is modified on the surface of the bacteria body to form an attachment layer through deposition.

3. The modified bacterium of claim 2, wherein the content of said metal compound on every 1*10⁸ bacteria is about 0.1 μg to 600 μg.

4. (canceled)

5. (canceled)

6. The modified bacterium of claim 2, wherein said bacterium body are selected from one or more of Staphylococcus aureus, Micrococcus urea, Diplococcus pneumoniae, Streptococcus pneumoniae, Diplococcus meningitidis, Streptococcus pyogenes, Streptococcus pneumoniae, Streptococcus lactis, Staphylococcus aureus, Staphylococcus albus, Staphylococcus citreus, Escherichia coli, Salmonella, Yersinia pestis, Bacillus dysenteriae suis, Pasteurella multocida, Corynebacterium diphtheriae, Mycobacterium tuberculosis Bacillus, Lactobacillus bifidum, Bacillus aceticus, Corynebacterium, Helicobacter pylori, Vibrio comma and Vibrio cholera.

7. (canceled)

8. (canceled)

9. The modified bacterium of claim 2, wherein said bacterium body is selected from one or more of Salmonella, Staphylococcus aureus, Escherichia coli, Lactobacillus, attenuated strains of Salmonella, attenuated strains of Staphylococcus aureus, attenuated strains of Escherichia coli and attenuated strains of Lactobacillus.

10. The modified bacterium of claim 2, wherein said bacterium body is a wild type bacterium, a genetically engineered bacterium, an attenuated strain, a living bacterium and/or an inactivated bacterium.

11. (canceled)

12. The modified bacterium of claim 10, wherein said living bacterium or inactivated bacterium comprises attenuated bacteria.

13. (canceled)

14. (canceled)

15. (canceled)

16. (canceled)

17. (canceled)

18. (canceled)

19. (canceled)

20. (canceled)

21. (canceled)

22. (canceled)

23. The modified bacterium of claim 2, wherein said metal compound is selected from one or more of zinc carbonate, calcium carbonate, copper carbonate, magnesium carbonate; zinc hydroxide, iron hydroxide, copper hydroxide, manganese hydroxide, magnesium hydroxide, zinc sulfide, copper sulfide, manganese sulfide;
zinc phosphate, calcium phosphate, copper phosphate, iron phosphate, magnesium phosphate and manganese phosphate.

24. (canceled)

25. The modified bacterium of claim 2, wherein said metal compound is manganese hydroxide, manganese dioxide and/or manganese sulfide.

26. (canceled)

27. A manganese-biomineralized bacterium comprising a bacterium body and a poorly soluble or slightly soluble manganese-containing compound attached to the surface of the bacterium body, wherein said poorly insoluble or slightly soluble manganese-containing compound is one or more of manganese hydroxide, manganese dioxide and manganese sulfide.

28. (canceled)

29. (canceled)

30. (canceled)

31. The manganese-biomineralized bacterium of claim 27, wherein said bacterium body are selected from one or more of Staphylococcus aureus, Micrococcus urea, Diplococcus pneumoniae, Streptococcus pneumoniae, Diplococcus meningitidis, Streptococcus pyogenes, Streptococcus pneumoniae, Streptococcus lactis, Staphylococcus aureus, Staphylococcus albus, Staphylococcus citreus, Escherichia coli, Salmonella, Yersinia pestis, Bacillus dysenteriae suis, Pasteurella multocida, Corynebacterium diphtheriae, Mycobacterium tuberculosis Bacillus, Lactobacillus bifidum, Bacillus aceticus, Corynebacterium, Helicobacter pylori, Vibrio comma and Vibrio cholerae.

32. (canceled)

33. (canceled)

34. The manganese-biomineralized bacterium of claim 27, wherein said bacterium body is a living bacterium, an inactivated bacterium a wild type bacterium, a genetically engineered bacterium and/or an attenuated strain.

35. (canceled)

36. A composition comprising the modified bacterium of claim 2, and optionally a pharmaceutically acceptable carrier, wherein said modified bacterium comprises a bacterium body and a poorly soluble or insoluble biologically acceptable metal compound modified on the surface of the bacterium body, and the pH value of the composition is 0-14.

37. A modified bacterial freeze-dried powder, comprising the modified bacterium of claim 2, and an additive.

38. The modified bacterial freeze-dried powder of claim 37, wherein said additive is selected from one or more of the following: binders, fillers, disintegrants, lubricants, wine, vinegar, decoction, ointment bases, cream bases, preservatives, antioxidants, flavoring agents, fragrances, co-solvents, emulsifiers, solubilizers, osmotic pressure regulators, colorants, sucrose, mannose, a-D-mannopyranose, trehalose, inositol, raffinose, inulin, dextran, maltodextrin, maltopolysaccharide, sucrose octasulfate, heparin and 2-hydroxypropyl-β-cyclodextrin

39. (canceled)

40. (canceled)

41. (canceled)

42. (canceled)

43. (canceled)

44. A manganese-biomineralized bacterial freeze-dried powder, comprising the manganese-biomineralized bacterium of claim 27, and an additive.

45. The manganese-biomineralized bacterial freeze-dried powder of claim 44, wherein said additive is selected from one or more of sugars/polyols, polymers, anhydrous solvents, surfactants, amino acids, salts, amines binders, fillers, disintegrants, lubricants, wine, vinegar, decoction, ointment bases, cream bases, preservatives, antioxidants, flavoring agents, fragrances, co-solvents, emulsifiers, solubilizers, osmotic pressure regulators, colorants, sucrose, mannose, a-D-mannopyranose, trehalose, inositol, raffinose, inulin, dextran, maltodextrin, maltopolysaccharide, sucrose octasulfate, heparin and 2-hydroxypropyl-β-cyclodextrin

46. (canceled)

47. (canceled)

48. (canceled)

49. (canceled)

50. (canceled)

51. A preparation method of the modified bacterium of claim 2, comprising the following steps:
S1: preparation of a bacterial suspension;
S2: adding a soluble metal salt solution to said bacterial suspension, and
S3: adding an easily soluble aqueous hydroxide solution, aqueous carbonate solution or aqueous phosphate solution until the pH is 8-12, or adding an easily soluble aqueous sulfide solution to react to prepare the modified bacterium.

52. (canceled)

53. (canceled)

54. (canceled)

55. The preparation method of claim 51, wherein in the step S2, a soluble metal salt solution is added to the bacterial suspension at an amount of 0.2-13.5 mmol of metal ions for every 1 billion bacteria, wherein said soluble metal salt solution is a permanganate solution.

56. (canceled)

57. (canceled)

58. (canceled)

59. (canceled)

60. (canceled)

61. A method for preventing and/or treating tumors, comprising administering to a subject in need thereof an effective amount of the modified bacterium of claim 2.

62. A method for preventing and/or treating tumors, comprising administering to a subject in need thereof an effective amount of modified bacterial freeze-dried powder of claim 37.
